(19) United States
(12) Patent Application Publication
(10) Pub. No.: US 2023/0285733 A1
(43) Pub. Date: Sep. 14, 2023

(54) PRIME TUBE CONFIGURATIONS FOR SYRINGE

(71) Applicant: Bayer HealthCare LLC, Whippany, NJ (US)

(72) Inventors: Kevin Cowan, Allison Park, PA (US); Michael Spohn, Fenelton, PA (US); Arthur Uber, III, Pittsburgh, PA (US); John Haury, Sewickley, PA (US); Joseph Kent, Pittsburgh, PA (US)

(21) Appl. No.: 18/019,939

(22) PCT Filed: Aug. 12, 2021
(86) PCT No.: PCT/US2021/045689

§ 371 Date: Feb. 6, 2023

Related U.S. Application Data
(60) Provisional application No. 63/065,095, filed on Aug. 13, 2020.

(51) Int. Cl. A61M 39/08; A61M 39/20; A61M 39/24; A61M 5/31

(52) U.S. Cl. CPC A61M 39/08; A61M 39/20; A61M 39/24; A61M 5/3146; A61M 2039/242

(57) ABSTRACT

A prime tube includes a sidewall defining an internal chamber having an expandable volume, a connecter associated with a proximal end of the sidewall and configured to reversibly engage an outlet of a fluid reservoir containing a medical fluid, and a closure associated with a distal end of the sidewall, the closure permeable to air and substantially impermeable to the medical fluid. The expandable volume of the internal chamber is configured to increase as the medical fluid enters the internal chamber.

### CROSS-REFERENCE TO RELATED APPLICATIONS

This application claims the benefit of U.S. Provisional Patent Application No. 63/065,095, filed on Aug. 13, 2020, the disclosure of which is hereby incorporated by reference in its entirety.

### BACKGROUND OF THE DISCLOSURE

### Field of the Disclosure

The present disclosure relates generally to prime tubes for syringes and/or pumps, such as syringes used in a fluid injector system for injecting a contrast media for a contrast enhanced imaging procedure. Specifically, the present disclosure relates to prime tubes for use in priming and/or purging air from a reservoir after filling the reservoir and prior to performance of an injection protocol where the prime tubes assist in determining when the air is purged from the reservoir.

### Description of Related Art

Powered fluid injector systems are widely used in medical imaging procedures such as angiography, computed tomography (CT) and nuclear magnetic resonance (NMR)/magnetic resonance imaging (MRI). Such injector systems provide precision and accuracy of fluid delivery beyond that which is achievable with a manual syringe, and furthermore may offer many safety features to prevent harm to the patient during an injection procedure.

During some injection procedures, it is imperative that no air be injected into the patient. Air may be present in the syringes or reservoir of a fluid injector system as packaged from the manufacturer. Additionally, air may accumulate in the syringes or reservoirs during automatic or manual filling of the syringes from one or more bulk fluid sources. Any air present in the volume must be purged prior to an injection procedure to avoid causing an air embolism.

For example, after the syringe is filled with fluid, a tubing set may be connected to the discharge outlet of the syringe, and the system may be actuated to prime the system by ejecting the air through the tubing, until the syringe and tubing are filled only with air. While this technique may be effective in purging air from the tubing connected to the syringe, dispensing fluid from the end of the tubing may cause contamination or fouling of system components and may lead to spills in the injection suite that may be a safety hazard and must be cleaned. Furthermore, in some cases it might not be clear to the technologist when the syringe has been fully primed, for example, visualization of air in the syringe may be difficult in low light or at distances, potentially leading to the assumption that a syringe is primed.

Therefore, new prime tube configurations that retain the priming fluids and are readily visualized to ensure priming before an injection are needed.

### SUMMARY OF THE DISCLOSURE

In view of the foregoing, there exists a need for devices, systems, and methods for improved priming of syringes and fluid injector systems. Embodiments of the present disclosure are directed to a prime tube for use with a fluid injector. The prime tube includes a sidewall defining an internal chamber having an expandable volume, a connecter associated with a proximal end of the sidewall and configured to reversibly engage an outlet of a fluid reservoir containing a medical fluid, and a closure associated with a distal end of the sidewall, the closure permeable to air and substantially impermeable to the medical fluid. The expandable volume of the internal chamber is configured to increase as the medical fluid enters the internal chamber.

In some embodiments, the sidewall includes at least one bellows. Each of the at least one bellows is configured to transition from a contracted state to an expanded state in response to an increase in fluid pressure within the internal chamber. The volume defined by the internal chamber is greater with the at least one of the at least one bellows in the expanded state than in the contracted state. In some embodiments, the at least one bellows is stable in both the contracted state and the expanded state. An axial length of the sidewall may be greater with the at least one bellows in the expanded state than in the contracted state.

In some embodiments, the sidewall includes an elastomeric material to expand the expandable volume in response to increased fluid pressure within the internal chamber.

In some embodiments, the sidewall may be configured to transition from a contracted state to an expanded state in response to an increase in fluid pressure within the internal chamber. In the contracted state, a distal portion of the sidewall is rolled over a proximal portion of the sidewall. In the expanded state, the distal portion of the sidewall is at least partially unrolled from the proximal portion of the sidewall. The volume defined by the internal chamber is greater with the sidewall in the expanded state than in the contracted state. In some embodiments, in the contracted state, an inner surface of the distal portion of the sidewall faces an inner surface of the proximal portion of the sidewall. In some embodiments, at least a portion of the sidewall is configured to invert in response to an increase in fluid pressure within the internal chamber to increase the volume of the internal chamber.

In some embodiments, the sidewall may be in a flaccid or bent configuration in the absence of fluid flow through the internal chamber and in a rigid, extended configuration when fluid flows into the internal chamber.

In some embodiments, the sidewall may be in a rolled, coiled configuration in the absence of fluid flow through the internal chamber and in an unrolled, extended configuration when fluid flows into the internal chamber.

In some embodiments, the prime tube may further include a check valve associated with the proximal end of the sidewall and configured to prevent fluid flow out of the proximal end. In some embodiments, the closure may include a high crack pressure valve.

In some embodiments, the closure includes a porous material. For example, the closure may define at least one aperture having a cross-sectional area sized to allow passage of air and substantially prohibit passage of a medical fluid through the at least one aperture.

In some embodiments, the at least one aperture may be configured to emit an audible sound when air flows through the at least one aperture.

Other embodiments of the present disclosure are directed to a prime tube for use with a fluid injection. The prime tube includes a sidewall defining an internal chamber, a connecter associated with a proximal end of the sidewall and configured to reversibly engage an outlet of a fluid reservoir containing a medical fluid, and a shuttle member configured to slide within the internal chamber in response to the medical fluid flowing into the internal chamber. The sidewall may be rigid so as to not deform in response to fluid pressure below a predetermined threshold. In some embodiments, at least one of the shuttle member and the sidewall defines an air passageway configured to allow air to flow distally past the shuttle member without causing the shuttle member to slide within the internal chamber. In some embodiments, the shuttle member may include a plug configured to form an interference fit with the outlet of the fluid reservoir such that the plug is dislodged from the outlet of the fluid reservoir at a predetermined fluid pressure. The plug may have an outside diameter sufficiently large to prevent a patient administration line from being attached to the outlet of the fluid reservoir while the plug is lodged in the outlet.

In some embodiments, the prime tube may include a cap having a proximal end configured to engage the outlet of the fluid reservoir and a distal end configured to engage the shuttle member. The shuttle member may be configured to be at least partially recessed within the outlet of the fluid reservoir prior to a priming operation.

In some embodiments, the prime tube further includes at least one engagement feature on the prime tube configured to retain the sidewall to the outlet of the fluid reservoir. The shuttle member, in an initial position prior to a priming operation, may lock the at least one engagement feature to the outlet of the fluid reservoir to prohibit removal of the prime tube from the fluid reservoir when the shuttle member is in the initial position. Upon distal movement of the shuttle member within the internal chamber to a second primed position, the at least one engagement feature may be unlocked from the outlet of the fluid reservoir to allow removal of the prime tube. In some embodiments, the shuttle member may include a tip configured to extend distally from a distal end of the prime tube when the shuttle member is moved to the second primed position.

In some embodiments, the shuttle member includes a porous material permeable to air and impermeable to the medical fluid. For example, the shuttle member may define at least one aperture having a cross-sectional area sized to allow passage of air and substantially prohibit passage of a medical fluid.

In some embodiments, the sidewall includes at least one indicator to indicate a distance traveled by the shuttle member corresponding to a fluid fill level of the internal chamber of the prime tube. In some embodiments, the sidewall is at least partially translucent or transparent such that the shuttle member is visible through the sidewall.

Other embodiments of the present disclosure are directed to a prime tube for use with a fluid injector system. The prime tube includes a sidewall defining an internal chamber having an expandable volume, and a connecter associated with a proximal end of the sidewall and configured to reversibly engage an outlet of a fluid reservoir containing a medical fluid. The sidewall is configured to transition from a contracted state to an expanded state in response to an increase in fluid pressure within the internal chamber. In the contracted state, a distal portion of the sidewall is rolled over a proximal portion of the sidewall. In the expanded state, the distal portion of the sidewall is at least partially unrolled from the proximal portion of the sidewall. The volume defined by the internal chamber is greater with the sidewall in the expanded state than in the contracted state. In the contracted state, an inner surface of the distal portion of the sidewall may face an inner surface of the proximal portion of the sidewall.

Other embodiments of the present disclosure are directed to a fluid injector system including at least one fluid reservoir configured for injecting a medical fluid and a prime tube. The prime tube includes a sidewall defining an internal chamber having an expandable volume, a connecter associated with a proximal end of the sidewall and configured to reversibly engage an outlet of the fluid reservoir. The system further includes at least one processor programmed or configured to determine a priming status of the prime tube.

In some embodiments, the at least one processor is programmed or configured to determine the priming status of the prime tube based on a measured fluid pressure in at least one of the prime tube and the fluid reservoir. For example, in some embodiments, the fluid injector system may further include an actuator for injecting the medical fluid from the at least one reservoir and the at least one processor may be programmed or configured to determine the priming status of the prime tube based on a measured current draw of the actuator. The at least one processor may be programmed or configured to determine the priming status of the prime tube based on a force reading on a motor of the fluid injector associated with delivering a fluid from the fluid reservoir.

In certain embodiments, the at least one processor may be programmed or configured to determine the priming status of the prime tube based on at least one of expansion of the prime tube and a shape change of the prime tube. In certain embodiments, the at least one processor may be programmed or configured to determine the priming status of the prime tube based on a sound emitted from the priming tube. In some embodiments, the sidewall includes an elastomeric material that expands the expandable volume in response to increased fluid pressure within the internal chamber.

In some embodiments, the sidewall may include at least one bellows. Each of the at least one bellows is configured to transition from a contracted state to an expanded state in response to an increase in fluid pressure within the internal chamber. The expandable volume defined by the internal chamber is greater with the at least one of the at least one bellows in the expanded state than in the contracted state.

In some embodiments, the sidewall is configured to transition from a contracted state to an expanded state in response to an increase in fluid pressure within the internal chamber. In the contracted state, a distal portion of the sidewall is rolled over a proximal portion of the sidewall. In the expanded state, the distal portion of the sidewall is at least partially unrolled from the proximal portion of the sidewall. The expandable volume defined by the internal chamber is greater with the sidewall in the expanded state than in the contracted state.

In some embodiments, the sidewall is in a rolled, coiled configuration in the absence of fluid flow through the internal chamber and in an unrolled, extended configuration when fluid flows through the internal chamber.

Other embodiments of the present disclosure are directed to a fluid injector system including at least one fluid reservoir configured for injecting a medical fluid, and a prime tube. The prime tube includes a sidewall defining an internal chamber, a shuttle member slidable within the internal chamber, and a connecter associated with a proximal end of the sidewall and configured to reversibly engage an outlet of the fluid reservoir. The fluid injector system further includes at least one processor programmed or configured to determine a priming status of the prime tube. The at least one processor may be programmed or configured to determine the priming status based on a position of the shuttle member within the internal chamber.

In some embodiments, the sidewall of the prime tube includes at least one indicator corresponding to a fluid fill level of the internal chamber and the at least one processor may be programmed or configured to determine the priming status based on a position of the shuttle member relative to the at least one indicator.

In some embodiments, the prime tube further includes a cap having a proximal end configured to engage the outlet of the fluid reservoir and a distal end configured to engage the shuttle member. The shuttle member may be configured to be at least partially recessed within the outlet of the fluid reservoir prior to a priming operation. In some embodiments, the prime tube further includes at least one engagement feature on the prime tube configured to retain the sidewall to the outlet of the fluid reservoir. The shuttle member, in an initial position prior to a priming operation, locks the at least one engagement feature to the outlet of the fluid reservoir to prohibit removal of the prime tube from the fluid reservoir when the shuttle member is in the initial position. Upon distal movement of the shuttle member within the internal chamber to a second primed position, the at least one engagement feature is unlocked from the outlet of the fluid reservoir to allow removal of the prime tube.

Further aspects or examples of the present disclosure are described in the following numbered clauses:
Clause 1. A prime tube for use with a fluid injector, the prime tube comprising: a sidewall defining an internal chamber having an expandable volume; a connecter associated with a proximal end of the sidewall and configured to reversibly engage an outlet of a fluid reservoir containing a medical fluid; and a closure associated with a distal end of the sidewall, the closure permeable to air and substantially impermeable to the medical fluid, wherein the expandable volume of the internal chamber is configured to increase as the medical fluid enters the internal chamber.
Clause 2. The prime tube according to clause 1, wherein the sidewall comprises at least one bellows, wherein each of the at least one bellows is configured to transition from a contracted state to an expanded state in response to an increase in fluid pressure within the internal chamber, wherein the volume defined by the internal chamber is greater with the at least one of the at least one bellows in the expanded state than in the contracted state.
Clause 3. The prime tube according to clause 1 or 2, wherein the at least one bellows is stable in both the contracted state and the expanded state.
Clause 4. The prime tube according to any of clauses 1 to 3, wherein an axial length of the sidewall is greater with the at least one bellows in the expanded state than in the contracted state.
Clause 5. The prime tube according to any of clauses 1 to 4, wherein the sidewall comprises an elastomeric material configured to expand the expandable volume in response to an increase in fluid pressure within the internal chamber.
Clause 6. The prime tube according to any of clauses 1 to 5, wherein the sidewall is configured to transition from a contracted state to an expanded state in response to an increase in fluid pressure within the internal chamber, wherein, in the contracted state, a distal portion of the sidewall is rolled over a proximal portion of the sidewall, wherein, in the expanded state, the distal portion of the sidewall is at least partially unrolled from the proximal portion of the sidewall, and wherein the volume defined by the internal chamber is greater with the sidewall in the expanded state than in the contracted state.
Clause 7. The prime tube according to any of clauses 1 to 6, wherein, in the contracted state, an inner surface of the distal portion of the sidewall faces an inner surface of the proximal portion of the sidewall.
Clause 8. The prime tube according to any of clauses 1 to 7, wherein at least a portion of the sidewall is configured to invert in response to an increase in fluid pressure within the internal chamber to increase the volume of the internal chamber.
Clause 9. The prime tube according to any of clauses 1 to 8, wherein the sidewall is in a flaccid or bent configuration in the absence of fluid flow through the internal chamber and in a rigid, extended configuration when fluid flows through the internal chamber.
Clause 10. The prime tube according to any of clauses 1 to 9, wherein the sidewall is in a rolled, coiled configuration in the absence of fluid flow through the internal chamber and in an unrolled, extended configuration when fluid flows through the internal chamber.
Clause 11. The prime tube according to any of clauses 1 to 10, further comprising: a check valve associated with the proximal end of the sidewall and configured to prevent fluid flow out of the proximal end.
Clause 12. The prime tube according to any of clauses 1 to 11, wherein the closure comprises a porous material.
Clause 13. The prime tube according to any of clauses 1 to 12, wherein the closure defines at least one aperture having a cross-sectional area sized to allow passage of air and substantially prohibit passage of a medical fluid through the at least one aperture.
Clause 14. The prime tube according to any of clauses 1 to 13, wherein the at least one aperture is configured to emit an audible sound when air flows through the at least one aperture.
Clause 15. The prime tube according to any of clauses 1 to 14, wherein the closure comprises a high crack pressure valve.
Clause 16. A prime tube for use with a fluid injection, the prime tube comprising: a sidewall defining an internal chamber; a connecter associated with a proximal end of the sidewall and configured to reversibly engage an outlet of a fluid reservoir containing a medical fluid; and a shuttle member configured to slide within the internal chamber in response to the medical fluid flowing into the internal chamber.
Clause 17. The prime tube according to clause 16, wherein the sidewall is rigid so as to not deform in response to fluid pressure below a predetermined threshold.
Clause 18. The prime tube according to clause 16 or 17, wherein at least one of the shuttle member and the sidewall defines an air passageway configured to allow air to flow distally past the shuttle member without causing the shuttle member to slide within the internal chamber.
Clause 19. The prime tube according to any of clauses 16 to 18, wherein the shuttle member comprises a plug configured to form an interference fit with the outlet of the fluid reservoir such that the plug is dislodged from the outlet of the fluid reservoir at a predetermined fluid pressure.
Clause 20. The prime tube according to any of clauses 16 to 19, wherein the plug has an outside diameter sufficiently large to prevent a patient administration line from being attached to the outlet of the fluid reservoir while the plug is lodged in the outlet.
Clause 21. The prime tube according to any of clauses 16 to 20, further comprising: a cap having a proximal end configured to engage the outlet of the fluid reservoir and a distal end configured to engage the shuttle member, wherein the shuttle member is configured to be at least partially recessed within the outlet of the fluid reservoir prior to a priming operation.
Clause 22. The prime tube according to any of clauses 16 to 21, further comprising: at least one engagement feature on the prime tube configured to retain the sidewall to the outlet of the fluid reservoir, wherein the shuttle member, in an initial position prior to a priming operation, locks the at least one engagement feature to the outlet of the fluid reservoir to prohibit removal of the prime tube from the fluid reservoir when the shuttle member is in the initial position, and wherein upon distal movement of the shuttle member within the internal chamber to a second primed position, the at least one engagement feature is unlocked from the outlet of the fluid reservoir to allow removal of the prime tube.
Clause 23. The prime tube according to any of clauses 16 to 22, wherein the shuttle member comprises a tip configured to extend distally from a distal end of the prime tube when the shuttle member is moved to the second primed position.
Clause 24. The prime tube according to any of clauses 16 to 23, wherein the shuttle member comprises a porous material permeable to air and impermeable to the medical fluid.
Clause 25. The prime tube according to any of clauses 16 to 24, wherein the shuttle member defines at least one aperture having a cross-sectional area sized to allow passage of air and substantially prohibit passage of a medical fluid.
Clause 26. The prime tube according to any of clauses 16 to 25, wherein the sidewall comprises at least one indicator to indicate a distance traveled by the shuttle member corresponding to a fluid fill level of the internal chamber of the prime tube.
Clause 27. The prime tube according to any of clauses 16 to 26, wherein the sidewall is at least partially translucent or transparent such that the shuttle member is visible through the sidewall.
Clause 28. A prime tube for use with a fluid injector system, the prime tube comprising: a sidewall defining an internal chamber having an expandable volume; and a connecter associated with a proximal end of the sidewall and configured to reversibly engage an outlet of a fluid reservoir containing a medical fluid, wherein the sidewall is configured to transition from a contracted state to an expanded state in response to an increase in fluid pressure within the internal chamber, wherein, in the contracted state, a distal portion of the sidewall is rolled over a proximal portion of the sidewall, wherein, in the expanded state, the distal portion of the sidewall is at least partially unrolled from the proximal portion of the sidewall, and wherein the volume defined by the internal chamber is greater with the sidewall in the expanded state than in the contracted state.
Clause 29. The prime tube according to clause 28, wherein, in the contracted state, an inner surface of the distal portion of the sidewall faces an inner surface of the proximal portion of the sidewall.
Clause 30. A fluid injector system, comprising: at least one fluid reservoir configured for injecting a medical fluid; a prime tube comprising: a sidewall defining an internal chamber having an expandable volume; and a connecter associated with a proximal end of the sidewall and configured to reversibly engage an outlet of the fluid reservoir; and at least one processor programmed or configured to determine a priming status of the prime tube.
Clause 31. The fluid injector system according to clause 30, wherein the at least one processor is programmed or configured to determine the priming status of the prime tube based on a measured fluid pressure in at least one of the prime tube and the fluid reservoir.
Clause 32. The fluid injector system according to clause 30 or 31, further comprising an actuator for injecting the medical fluid from the at least one reservoir, wherein the at least one processor is programmed or configured to determine the priming status of the prime tube based on a measured current draw of the actuator.
Clause 33. The fluid injector system according to any of clauses 30 to 32, wherein the at least one processor is programmed or configured to determine the priming status of the prime tube based on at least one of expansion of and a shape change of the prime tube.
Clause 34. The fluid injector system according to any of clauses 30 to 33, wherein the at least one processor is programmed or configured to determine the priming status of the prime tube based on a sound emitted from the priming tube.
Clause 35. The fluid injector system according to any of clauses 30 to 34, wherein the at least one processor is programmed or configured to determine the priming status of the prime tube based on a force reading on a motor of the fluid injector associated with delivering a fluid from the fluid reservoir.
Clause 36. The fluid injector system according to any of clauses 30 to 35, wherein the sidewall comprises at least one bellows, wherein each of the at least one bellows is configured to transition from a contracted state to an expanded state in response to an increase in fluid pressure within the internal chamber, wherein the expandable volume defined by the internal chamber is greater with the at least one of the at least one bellows in the expanded state than in the contracted state.
Clause 37. The fluid injector system according to any of clauses 30 to 36, wherein the sidewall comprises an elastomeric material configured to expand the expandable volume in response to an increase in fluid pressure within the internal chamber.
Clause 38. The fluid injector system according to any of clauses 30 to 37, wherein the sidewall is configured to transition from a contracted state to an expanded state in response to an increase in fluid pressure within the internal chamber, wherein, in the contracted state, a distal portion of the sidewall is rolled over a proximal portion of the sidewall, wherein, in the expanded state, the distal portion of the sidewall is at least partially unrolled from the proximal portion of the sidewall, and wherein the expandable volume defined by the internal chamber is greater with the sidewall in the expanded state than in the contracted state.
Clause 39. The fluid injector system according to any of clauses 30 to 38, wherein the sidewall is in a rolled, coiled configuration in the absence of fluid flow through the internal chamber and in an unrolled, extended configuration when fluid flows through the internal chamber.
Clause 40. A fluid injector system, comprising: at least one fluid reservoir configured for injecting a medical fluid; a prime tube comprising: a sidewall defining an internal chamber; a shuttle member slidable within the internal chamber; and a connecter associated with a proximal end of the sidewall and configured to reversibly engage an outlet of the fluid reservoir; and at least one processor programmed or configured to determine a priming status of the prime tube.
Clause 41. The fluid injector system according to clause 40, wherein the at least one processor is programmed or configured to determine the priming status of the prime tube based on a position of the shuttle member within the internal chamber.
Clause 42. The fluid injector system according to clause 40 or 41, wherein the sidewall of the prime tube comprises at least one indicator corresponding to a fluid fill level of the internal chamber, and wherein the at least one processor is programmed or configured to determine the priming status of the prime tube based on a position of the shuttle member relative to the at least one indicator.
Clause 43. The fluid injector system according to any of clauses 40 to 42, wherein the prime tube further comprises a cap having a proximal end configured to engage the outlet of the fluid reservoir and a distal end configured to engage the shuttle member, and wherein the shuttle member is configured to be at least partially recessed within the outlet of the fluid reservoir prior to a priming operation.
Clause 44. The fluid injector system according to any of clauses 40-43, wherein the prime tube further comprises at least one engagement feature on the prime tube configured to retain the sidewall to the outlet of the fluid reservoir, wherein the shuttle member, in an initial position prior to a priming operation, locks the at least one engagement feature to the outlet of the fluid reservoir to prohibit removal of the prime tube from the fluid reservoir when the shuttle member is in the initial position, and wherein upon distal movement of the shuttle member within the internal chamber to a second primed position, the at least one engagement feature is unlocked from the outlet of the fluid reservoir to allow removal of the prime tube.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1 is a perspective view of a fluid injector system according to an embodiment of the present disclosure;

FIG. 2A is a schematic view of a fluid injector system in accordance with an embodiment of the present disclosure;

FIG. 2B is a partial schematic view of the fluid injector system of FIG. 2A, shown with a prime tube attached to each syringe (various components of FIG. 2A are not shown in FIG. 2B for clarity);

FIG. 3 is a schematic view of a prime tube of FIG. 2B according to an embodiment of the present disclosure;

FIG. 4A is a perspective view of a prime tube according to an embodiment of the present disclosure;

FIG. 4B is an exploded view of the prime tube of FIG. 4A;

FIG. 4C is a side cross-sectional view of the prime tube of FIG. 4A in a contracted state;

FIG. 4D is a side cross-sectional view of the prime tube of FIG. 4A in an expanded state;

FIG. 4E is a graph of pressure over time for the prime tube of FIG. 4A;

FIG. 5A is a perspective view of a prime tube according to an embodiment of the present disclosure;

FIG. 5B is an exploded view of the prime tube of FIG. 5A;

FIG. 5C is a side cross-sectional view of the prime tube of FIG. 5A in a contracted state;

FIG. 5D is a side cross-sectional view of the prime tube of FIG. 5A in an expanded state;

FIG. 5E is a graph of pressure over time for the prime tube of FIG. 5A;

FIG. 6A is a perspective view of a prime tube according to an embodiment of the present disclosure;

FIG. 6B is an exploded view of the prime tube of FIG. 6A;

FIG. 6C is a side cross-sectional view of the prime tube of FIG. 6A in a contracted state;

FIG. 6D is a side cross-sectional view of the prime tube of FIG. 6A in an expanded state;

FIG. 6E is a graph of pressure over time for the prime tube of FIG. 6A;

FIG. 7A is a perspective view of a prime tube according to an embodiment of the present disclosure;

FIG. 7B is an exploded view of the prime tube of FIG. 7A;

FIG. 7C is a side cross-sectional view of the prime tube of FIG. 7A in a contracted state;

FIG. 7D is a side cross-sectional view of the prime tube of FIG. 7A in an expanded state;

FIG. 7E is a graph of pressure over time for the prime tube of FIG. 7A-7D;

FIG. 8A is a perspective view of a prime tube according to an embodiment of the present disclosure;

FIG. 8B is an exploded view of the prime tube of FIG. 8A;

FIG. 8C is a side cross-sectional view of the prime tube of FIG. 8A in an initial state;

FIG. 8D is a side cross-sectional view of the prime tube of FIG. 8A in a primed state;

FIG. 8E is a graph of pressure over time for the prime tube of FIG. 8A;

FIG. 9A is a perspective view of a prime tube according to an embodiment of the present disclosure;

FIG. 9B is an exploded view of the prime tube of FIG. 9A;

FIG. 9C is a side cross-sectional view of the prime tube of FIG. 9A in an initial state;

FIG. 10A is a perspective view of a prime tube according to an embodiment of the present disclosure;

FIG. 10B is an exploded view of the prime tube of FIG. 10A;

FIG. 10C is a side cross-sectional view of the prime tube of FIG. 10A;

FIG. 11A is a perspective view of a prime tube according to an embodiment of the present disclosure;

FIG. 11B is an exploded view of the prime tube of FIG. 11A;

FIG. 11C is a side cross-sectional view of the prime tube of FIG. 11A in an initial state;

FIG. 11D is a side cross-sectional view of the prime tube of FIG. 11A in a primed state;

FIG. 12A is a perspective view of a prime tube according to an embodiment of the present disclosure;

FIG. 12B is an exploded view of the prime tube of FIG. 12A;

FIG. 12C is a side cross-sectional view of the prime tube of FIG. 12A in an initial state;

FIG. 12D is a side cross-sectional view of the prime tube of FIG. 12A in a primed state;

FIG. 13 is a side cross-sectional view of a prime tube according to an embodiment of the present disclosure;

FIG. 14A is a side view of a prime tube according to an embodiment of the present disclosure;

FIG. 14B is a side view of the prime tube of FIG. 14A in a contracted state;

FIG. 14C is a side cross-sectional view of the prime tube of FIG. 14A in an expanded, primed state;

FIG. 15A is a side view of a prime tube in an initial state according to an embodiment of the present disclosure; and

FIG. 15B is a side view of the prime tube of FIG. 15A in a primed configuration.

Referring to the drawings in which like reference characters refer to like parts throughout the several views thereof, the present disclosure is generally directed to a prime tube for use with a syringe of a fluid injector system.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. Spatial or directional terms, such as “left”, “right”, “inner”, “outer”, “above”, “below”, and the like, are not to be considered as limiting as the invention can assume various alternative orientations. As used herein, the singular form of “a”, “an”, and “the” include plural referents unless the context clearly dictates otherwise. All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a component of a fluid delivery system such as a fluid reservoir, a syringe, an air suspension apparatus, or a fluid line, the term “distal” refers to a portion of said component nearest to a patient. When used in relation to a component of an injector system such as a fluid reservoir, a syringe, an air suspension apparatus, or a fluid line, the term “proximal” refers to a portion of said component nearest to the injector of the injector system (i.e. the portion of said component farthest from the patient). When used in relation to a component of a fluid delivery system such as a fluid reservoir, a syringe, an air suspension apparatus, or a fluid line, the term “upstream” refers to a direction away from the patient and towards the injector of the injector system. For example, if a first component is referred to as being “upstream” of a second component, the first component is located nearer to the injector than the second component is to the injector. When used in relation to a component of a fluid delivery system such as a fluid reservoir, a syringe, an air suspension apparatus, or a fluid line, the term “downstream” refers to a direction towards the patient and away from the injector of the fluid delivery system. For example, if a first component is referred to as being “downstream” of a second component, the first component is located nearer to the patient than the second component is to the patient. The terms “first”, “second”, and the like are not intended to refer to any particular order or chronology, but refer to different conditions, properties, or elements. The term “at least” is synonymous with “greater than or equal to”. The term “not greater than” is synonymous with “less than or equal to”.

It is to be understood that the disclosure may assume alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the examples disclosed herein are not to be considered as limiting.

While the devices, systems, and methods described herein are generally discussed in the context of an angiography (CV) injection system, other pressurized injection protocols, such as computed tomography (CT), ultrasound, positron emission tomography (PET), and magnetic resonance imaging (MRI) may also incorporate the various embodiments of the prime tubes described herein. Further, while many of the embodiments or prime tubes described herein are detailed in reference to a syringe or syringe based fluid injector, it should be understood that other powered injection systems, such as those including pumps such as a peristaltic pump, which require priming of a reservoir and/or a fluid tube set can be primed using various embodiments of the prime tubes herein.

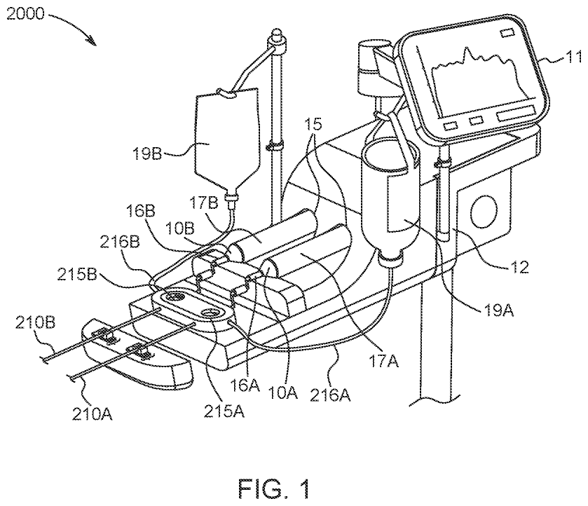

Referring to the drawings in which like reference characters refer to like parts throughout the several views thereof, the present disclosure is generally directed to prime tubes for syringes of fluid injector systems. Referring first to FIG. 1, an embodiment of a dual syringe angiography injector system 2000 is illustrated. The angiography injector system 2000 is configured for injection of two medical fluids through a first fluid path 210A for a medical fluid, such as an imaging contrast media for an angiography injection procedure, and a second fluid path 210B for a flushing fluid, such as saline or Ringer's lactate. The fluid paths 210A, 210B may be connected to respective outlets 16A, 16B, e.g., nozzles, of syringes 10A, 10B. The dual syringe angiography injector system 2000 may include an injector housing 12 having two syringe ports 15 configured to engage the syringes 10A, 10B. In some embodiments, the syringes 10A, 10B may be retained within corresponding pressure jackets 17A, 17B for example to prevent pressure-induced swelling and potential bursting of the syringes 10A, 10B.

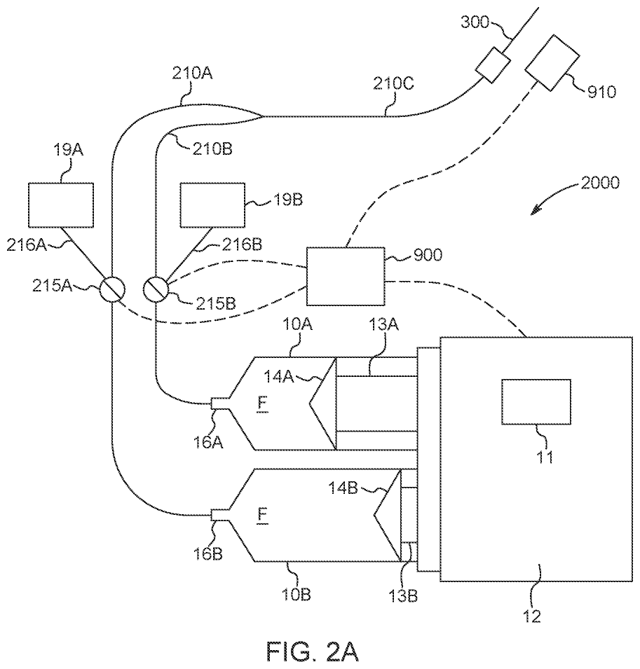

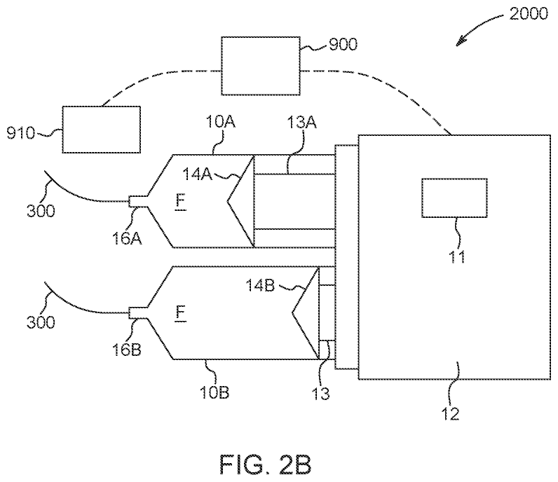

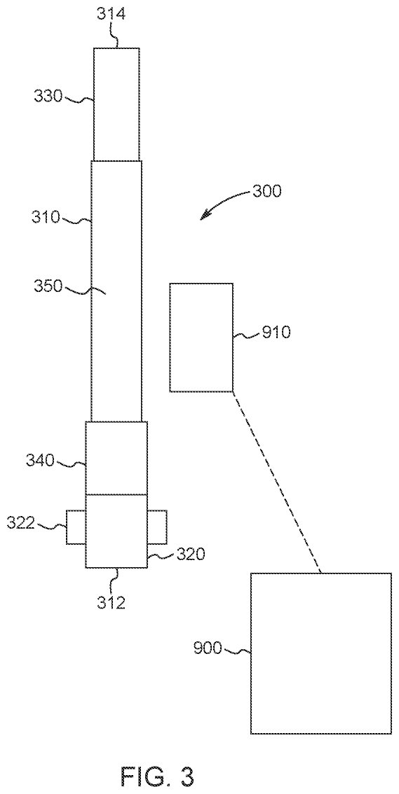

The fluid injector system 2000 may further include at least one graphical user interface (GUI) 11 through which an operator can view and control the status of an injection procedure. The GUI 11 may be in operative communication with a controller 900 (see FIGS. 2A-3) which sends and receives commands between and receives input from the GUI 11 and fluid injector system 2000.

With continued reference to FIG. 1, the dual syringe angiography injector system 2000 may further include bulk fluid containers 19A and 19B for filling and refilling the respective syringes 10A, 10B with imaging contrast media and flushing fluid, respectively. The bulk fluid containers 19A and 19B may be in selective fluid communication with the syringes 10A, 10B via respective bulk fluid paths 216A and 216B and bulk fluid valves 215A and 215B.

Further details and examples of suitable nonlimiting powered injector systems, including syringes, pressure jackets and pressure jacket retention mechanisms, tubing, shut-off valves, controllers, and air detectors, are described in U.S. Pat. Nos. 5,383,858; 7,553,294; 7,666,169; 8,945,051; 10,022,493; and 10,507,319, and International PCT Application Nos. PCT/US2013/061275; PCT/US2018/034613; PCT/US2020/049885; PCT/US2021/035273; PCT/US2021/029963; PCT/US2021/018523; PCT/US2021/037623; PCT/US2021/037574; and PCT/US2021/045298, the disclosures of which are hereby incorporated by reference in their entireties.

Referring now to FIG. 2A, a schematic diagram of the fluid injector system 2000 shown in FIG. 1 is illustrated. The injector system 2000 includes a piston 13A, 13B respectively associated with each of the syringes 10A, 10B and their corresponding pressure jackets 17A, 17B (see FIG. 1). Each of the pistons 13A, 13B is configured to drive a respective plunger 14A, 14B within a barrel of the respective syringe 10A, 10B. The controller 900 is operatively associated with the injector system 2000, for example to activate the pistons 13A, 13B to reciprocatively move the plungers 14A, 14B within the syringes 10A, 10B and thereby execute and halt an injection procedure. In a corresponding peristaltic pump system, the controller 900 would be configured to operate the rotors of the corresponding peristaltic pump. In particular, the controller 900 may include at least one processor programmed or configured to actuate the pistons 13A, 13B and various other components of the injector system 2000, such as one or more valves 215A, 215B, to take in and deliver medical fluids according to a programmed protocol for an injection procedure. The controller 900 may include computer readable media, such as memory, on which one or more injection protocols may be stored for execution by the at least one processor.

The controller 900 may be programmed or configured to execute a filling operation during which the piston 13A, 13B associated with each syringe 10A, 10B is withdrawn toward a proximal end of the syringe 10A, 10B to draw injection fluid F (e.g. imaging contrast media and flushing fluid) into the syringe 10A, 10B from the bulk fluid containers 19A, 19B. During such a filling operation, the controller 900 may be programmed or configured to selectively actuate the bulk fluid valves 215A and 215B to establish fluid communication between the respective syringes 10A, 10B and the bulk fluid containers 19A, 19B via the bulk fluid paths 216A and 216B to control filling of syringes 10A, 10B with an appropriate injection fluid F.

The controller 900 may be programmed or configured to execute a priming/purging operation to remove any air from the syringes 10A, 10B upon completion of the filling operation. Specific details of the priming/purging operation will be described herein in connection with the various embodiments of the prime tubes 300 shown in FIGS. 3-15B.

After the filling operation and priming operation, the controller 900 may be programmed or configured to execute a delivery operation during which the piston 13A, 13B associated with one or both of the syringes 10A, 10B is moved toward a distal end of the syringe to inject injection fluid F into the first fluid path 210A and the second fluid path 210B, respectively. The controller 900 may be programmed or configured to selectively actuate the bulk fluid valves 215A and 215B to establish fluid communication between the syringes 10A, 10B and the patient, via the fluid paths 210A, 210B. The first fluid path 210A and the second fluid path 210B ultimately merge into a patient fluid line 210C in fluid communication with the vasculature of the patient.

Referring now to FIG. 2B, the fluid injector system 2000 is shown with a prime tube 300 attached to each of the syringes 10A, 10B. The prime tubes 300 may be attached to the syringes 10A, 10B after a filling operation in which the syringes 10A, 10B are filled from the bulk fluid containers 19A and 19B and prior to an injection procedure. Other embodiments may be directed towards priming of at least a portion of a tubing set in preparation for a fluid injection procedure. For example, in one embodiment, the prime tube configurations described herein may be attached to a distal end of a tubing set (as shown in FIG. 2A), such as a multi-use portion of a tubing set and used to ensure priming of air from the syringe and the multi-use portion of the tubing set. In other embodiments, the prime tube may be attached to a distal end of a newly installed single patient tubing set (attached to the multi-use tube set) to ensure priming of the single patient tubing set prior to attaching to a patient catheter. The fluid paths 210A, 210B may be disconnected from the syringes 10A, 10B to allow for connection of the prime tubes 300. With the prime tubes 300 connected to the syringes 10A, 10B, the fluid injector system 2000 may be actuated to prime/purge the syringes 10A, 10B by extending the pistons 13A, 13B distally to eject air from the syringes 10A, 10B into the associated prime tubes 300. In some embodiments, the controller 900 may be programmed or configured to extend each of the pistons 13A, 13B a predetermined distance, corresponding to a predetermined fluid volume, to eject the predetermined air/fluid volume from each syringe 10A, 10B into the associated prime tubes 300. The predetermined air/fluid volume may be selected to ensure that all (or substantially all) of the air in the syringes 10A, 10B is ejected into the prime tubes 300. For example, the predetermined air/fluid volume may be selected based on empirical data of the maximum volume of air within syringes after a fill operation. The predetermined air/fluid volume may include a factor of safety to ensure that even an abnormally high amount of air will be fully ejected from the syringe during the priming/purging operation. As the predetermined air/fluid volume is typically larger than the actual volume of air present in the syringes 10A, 10B, some amount of the medical fluid F will be ejected into the prime tubes 300 along with the volume of air. Once the priming operation is complete, the prime tubes 300 may be disconnected from the syringes 10A, 10B and the fluid paths 210A, 210B may be reconnected in the arrangement shown in FIG. 2A in preparation for an injection procedure. The prime tubes 300 may be configured to retain the medical fluid F after the prime tubes 300 are disconnected from the syringes 10A, 10B to prevent spills and leaks. In some embodiments, prime tubes 300 may be discarded once disconnected from syringes 10A, 10B.

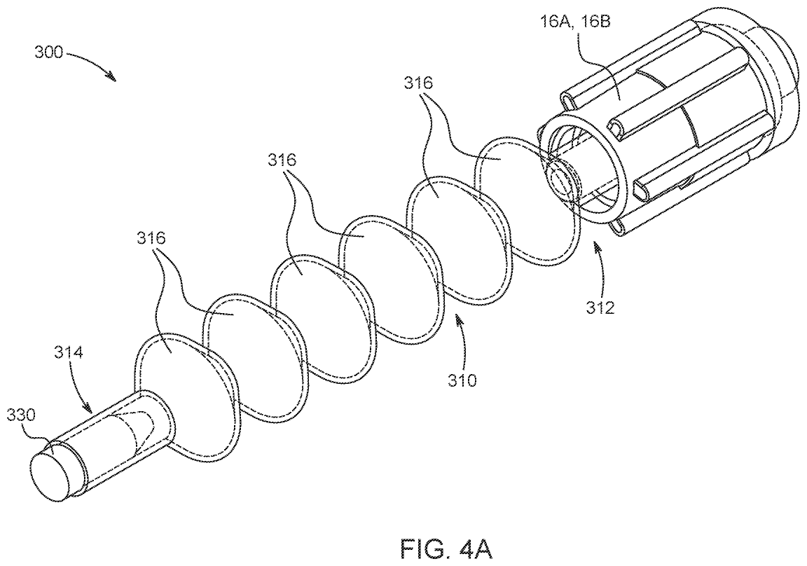

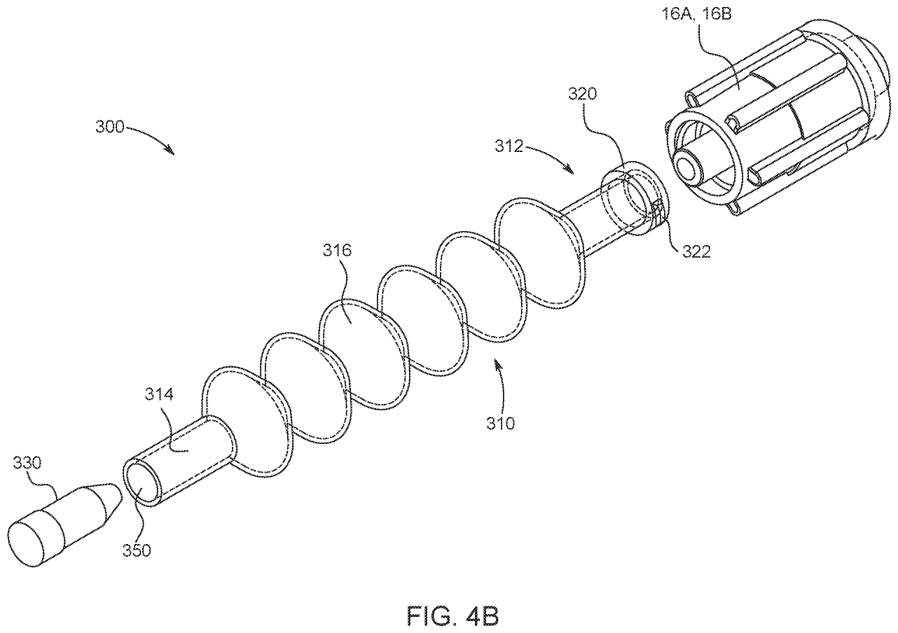

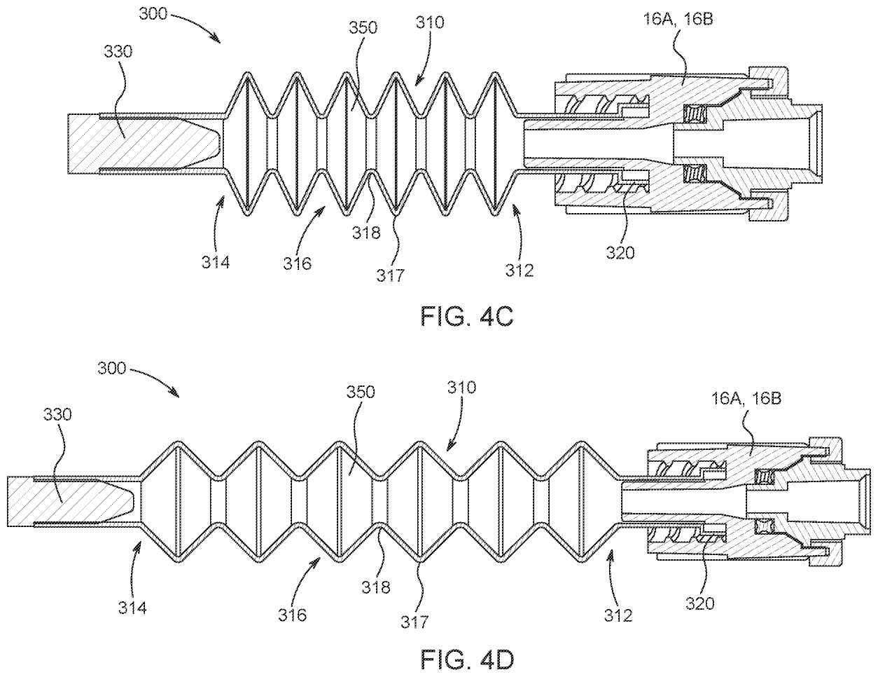

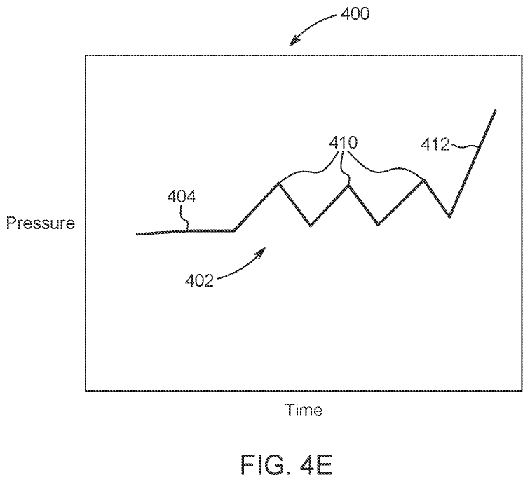

In some embodiments, the fluid injector system 2000 may include one or more priming status sensors 910 associated with the prime tubes 300 and/or the syringes 10A, 10B. The priming status sensors 910 may be in communication with the controller 900 and may be configured to detect a priming status, e.g. whether the syringes 10A, 10B have been primed to remove all air therefrom, based on observation and/or measurement of air and fluid in the prime tubes 300. In various embodiments, the priming status sensors 910 may include optical sensors (e.g. cameras), strain gauges, microphones, ammeters, limit switches, pressure transducers, or any other variety of sensor capable of detecting a property of the system 2000 that can be used to determine the priming status. Further details of the priming status sensors 910 will be provided in the context of particular embodiments of the prime tubes 300 as described herein with reference to FIGS. 4-15B.

Referring now to FIG. 3, in some embodiments, the prime tube 300 may include a sidewall 310 having a proximal end 312 and a distal end 314 and defining an internal chamber 350. The proximal end 312 of the sidewall 310 may include a connector 320 configured to reversibly engage the outlet 16A, 16B of the associated fluid reservoir, such as syringe 10A, 10B. In some embodiments, the connector 320 may include one or more engaging lugs 322 having a profile complementary to an inner threading of the outlet 16A, 16B of the syringe 10A, 10B. In some embodiments, the one or more engaging lugs 322 may be configured to release from the outlet 16A, 16B of the syringe 10A, 10B at a predetermined fluid pressure within the prime tube 300. In some embodiments, the connector 320 may be a female luer connector. In other embodiments, the connector may be a male or female portion of the connector and configured to interface/connect with the corresponding complementary female or male connector as described in International PCT Application No. PCT/US2021/018523.

In some embodiments, the prime tube 300 may include a closure 330 associated with the distal end 314 of the sidewall 310. The closure 330 may be permeable to air and substantially impermeable to liquids, including the medical fluid F. For example, according to various embodiments, the closure 330 may be a porous material, such as a material having at least one aperture having a cross-sectional area sized to allow to allow passage of air and substantially prohibit passage of a medical fluid through the at least one aperture. As such, during a priming operation of the syringes 10A, 10B, the air ejected from the syringes 10A, 10B by pressurization of the syringe contents may flow out of the prime tube 300 via the closure 330, while any medical fluid F ejected from the syringe 10A, 10B is retained within the internal chamber 350. Due to the differences in compressibility of a gas, such as air, and a liquid, such as the medical fluid F (e.g., contrast or saline) pressure build-up in the prime tube 300 substantially increases after all the air has been ejected from the syringe and liquid is ejected into the prime tube 300. According to various embodiments, the pressure differential between the air and the liquid medical fluid may be used to determine the prime status of the syringe. In some embodiments, the closure 330 may be made from a hydrophobic medical grade plastic, such as those commercially available under the trade names Gore-Tex® and Porex®, which generally allow passage of gas but not liquid therethrough. In some embodiments, the closure 330 may be a solid material defining one or more apertures having a sufficient cross-sectional area to allow the passage of air out of the prime tube 300, but an insufficient cross-sectional area to allow the passage of the medical fluid F. In some embodiments, the closure 330 may include an absorbent material, such as cotton or other fibrous material, to absorb the medical fluid F while allowing air to pass out of the prime tube 300. In some embodiments, the closure 330 may be a solid member impermeable to all fluids, e.g. air and medical fluids, such that all fluids injected into the prime tube 300 from the syringe 10A, 10B are retained within the internal chamber 350.

In some embodiments, the closure 330 may include a crack pressure valve, such as a high crack pressure valve, configured to open in response to a predetermined fluid pressure. A such, the closure 330 may remain closed as pressure builds within the prime tube 300, and the closure 330 may open at the predetermined fluid pressure to allow air and/or fluid out of the prime tube 300.

In some embodiments, the internal chamber 350 may define an expandable volume. That is, the volume of the internal chamber 350 may increase as the sidewall 310 stretches, unrolls, unfolds, or otherwise changes shape. In such embodiments, the sidewall 310 may be made from a stretchable, foldable, and/or resilient material. In some embodiments, the expandable volume of the internal chamber 350 increases as fluid enters the internal chamber 350 such that the internal chamber 350 adopts the volume of the fluid ejected from the syringe 10A, 10B. In some embodiments, the internal chamber 350 may be configured to expand or become rigid in response to an increase in fluid pressure as fluid is ejected into the internal chamber 350.

In some embodiments, the sidewall 310 may be biased toward a contracted state, e.g. a state in which the prime tube 300 is supplied and initially connected to the syringe 10A, 10B wherein the internal chamber 350 has a minimum volume. According to certain configurations, once the internal chamber 350 expands due to an increase in fluid pressure, such fluid pressure must be maintained or the internal chamber 350 will have a tendency to return, at least partially, to the contracted state and eject the fluid back out of the connector 320. According to these configurations, a one-way valve 340, such as a check valve, may be disposed adjacent to or integral with the connector 320 of the prime tube 300 to prevent fluid flow out of the prime tube 300 when fluid pressure is relieved, e.g. when the prime tube 300 is disconnected from the syringe 10A, 10B. Thus, inadvertent spills or leakage of the medical fluid when the prime tube 300 is removed from the syringe are avoided.

In some embodiments, the sidewall 310 may be stable in both the contracted state and the expanded state, in that the internal chamber 350 is not biased toward either the contracted state or an expanded state. In such embodiments, relief of fluid pressure from the internal chamber 350, such as when the prime tube 300 is disconnected from the syringe 10A, 10B, does not result in the internal chamber 350 returning to the contracted state. As such, the one-way valve 340 may not be required to prevent fluid flow back out of the prime tube 30. However, the one-way valve 340 may still be provided to prevent inadvertent discharge of fluid, such as if the technologist squeezes the sidewall 310 during removal of the prime tube 300 from the syringe 10A, 10B or accidently tips the prime tube 300 so that gravity causes the fluid therein to flow through the connector 320.

The shape change of the sidewall 310 due to the expansion of the internal chamber 350 may be used as an indicator that the priming/purging operation of the syringe 10A, 10B has been completed. The shape change of the sidewall 310 may be detected by priming status sensor 910, and the priming status sensor 910 may send an output signal to controller 900 indicating the priming status of syringes 10A, 10B. In other embodiments, the technician may visually or audibly determine that a priming operation is completed.

In some embodiments, the sidewall 310 may be substantially rigid, meaning that the sidewall does not substantially deform under the pressure associated with an injection procedure, e.g. 1200 psi for a CV procedure.

In some embodiments, the internal chamber 350 may have a volume sufficient to hold the predetermined fluid volume ejected from the associated syringe 10A, 10B during the syringe priming/purging operation. In some embodiments, the volume defined by the internal chamber 350 may range from 5 mL to 30 mL and in other embodiments, from 5 mL to 10 mL. In specific embodiments, the volume should be sufficient to account for cases when more than one prime sequence is required, for example when air bubbles are still observed in the system. In certain embodiments, a prime operation may include two injection sequences, a first forceful flow rate with small ejected volumes to remove the majority of air from system and then a second slower flow rate with larger flow volumes, optionally accompanied by tapping or vibrations, to remove small air bubbles.

To perform a priming/purging operation to expel air from the syringe 10A, 10B, the syringe 10A, 10B may be held in a substantially upright vertical position with the outlet 16A, 16B at the highest point, the syringe 10A, 10B may be primed, e.g., the pistons 13A, 13B moved in the distal direction to expel a portion of the contents of the syringe (i.e., air and medical fluid F), to purge any air from the interior of the syringe 10A, 10B. In the vertical position, a majority of the air in the syringe 10A, 10B rises to the distal end of the syringe adjacent the outlet 16A, 16B due to buoyancy of air in the medical fluid F. As the syringe 10A, 10B is primed, the air at the distal end of the syringe 10A, 10B is expelled through the outlet 16A, 16B and enters the internal chamber 350 of the prime tube 300. During the priming operation, air may be dislodged from the various interior surfaces of the syringe and the surface of the plunger to ensure that all air is primed. Examples of such methods for dislodging air bubbles (including microbubbles) from an interior surface or plunger surface are described in International PCT Application Publication No. WO 2019/204617, the disclosure of which is incorporated herein, for example by placing the syringe contents under an at least partial vacuum to coalesce the small air bubbles into larger bubbles and/or vibrating, knocking, or otherwise impacting the syringe or piston/plunger assembly to dislodge the air bubbles.

Referring now to FIGS. 4A-15B, various embodiments of the prime tube 300 are illustrated. Components in the embodiments shown in FIGS. 4A-15B having the same reference numerals as the embodiment of FIG. 3 indicate similar and/or functionally equivalent components. Referring first to FIGS. 4A-4D, an embodiment of a prime tube 300 includes an expanding sidewall 310 having at least one bellows 316. The at least one bellows 316 are configured to expand and contract longitudinally to increase the volume of the internal chamber extending longitudinally between the proximal end 312 and the distal end 314 of the sidewall 310. The at least one bellows 316 include alternating wider diameter sections 317 and narrow diameter sections 318 (see FIG. 4D). Initially, the at least one bellows 316 is provided in a contracted state (see FIG. 4C) in which the distance between the proximal-most wider diameter section 317 and the distal-most wider diameter section 317 is minimized. In the contracted state of FIG. 4C, the length of the sidewall 310 shorter than in the expanded state shown in FIG. 4D and the interior volume of the internal chamber 350 is minimized. Consequently, the volume defined by the internal chamber 350 is greater in the expanded state.

With continued reference to FIGS. 4A-4D, the closure 330, in some embodiments in the form of a porous plug formed of a porous hydrophobic polymer or other material as described herein and is substantially permeable to air and impermeable to liquid, is inserted or otherwise connected to the distal end 314 of the prime tube 300. The closure 330 may be configured to allow air or other gas to pass through the pores of the material while preventing a medical fluid F from exiting the internal chamber 350. It should be recognized that the position of the porous plug may be located elsewhere on the prime tube 300 while still allowing air to escape the prime tube 300 and retain liquid in the prime tube 300.

During a priming/purging operation of the syringe 10A, 10B, air ejected from the syringe 10A, 10B passes through the internal chamber 350 and exits through the closure 330 at the distal end 314. Due to the compressibility of air, the configuration of the at least one bellows 316 remains substantially unchanged and in the contracted state. As the air is continued to be primed, eventually some medical fluid F pushes the remaining air from the syringe 10A, 10B into the prime tube 300 and some amount of medical fluid F itself enters the prime tube 300. The medical fluid F moves through the at least one bellows 316 and contacts the porous plug of the closure 330, through which the medical fluid F is unable to pass. Consequently, fluid pressure builds in the internal chamber 350 and the at least one bellows 316 begin to expand from the compressed state to the extended state (compare FIG. 4C to FIG. 4B).

Expansion of the bellows 316 of the sidewall 310 may be visualized by the technologist to confirm that substantially all air has been purged from the syringe 10A, 10B and that the syringe 10A, 10B is primed and ready for use. In some embodiments, the priming status sensor 910 (see FIGS. 2B and 3) may include a detector for image recognition, such as a camera as described in U.S. Pat. No. 10,201,666, the disclosure of which is incorporated by this reference. Based on an output signal from the priming status sensor 910, the controller 900 may be programmed or configured to determine that the prime tube 300 is in the expanded state and the syringe 10A, 10B is primed of air. The processor 900 may be configured to prevent a fluid injection procedure until it receives indication that the syringe 10A, 10B has been successfully primed. Once a successful prime has been established as determined by the controller 900 and/or the technologist, the prime tube 300 may be removed and discarded according to hospital protocol, and a suitably primed patient catheter line may be installed on the syringe 10A, 10B. It should be noted that while the prime tube embodiments described herein are utilized to ensure priming of air from a syringe, the various embodiments of the prime tube may also be used to ensure priming of air from a tubing set as well as the syringe. According to these embodiments, the prime tube may be placed at the distal end of the tubing set (as shown in FIG. 2A) and used in a similar manner to determine with all air has been effectively ejected from the internal volume of the tubing set, and in certain embodiments, form the corresponding syringe(s) and/or peristaltic pump(s) mechanism attached to the proximal end(s) of the tubing set.

With continued reference to FIGS. 4A-4D, in some embodiments, each of the bellows 316 may be configured to emit an audible sound, such as a pop, when transitioning from the contracted state to the expanded state. The technologist may use the sound as an indication that substantially all air has been purged from the syringe 10A, 10B and the syringe 10A, 10B is primed and ready for use. Additionally, the priming status sensor 910 may include a microphone configured to detect the sound and send a signal to the processor 900, which may in turn determine that the syringe 10A, 10B has been primed based on the signal. For example, the technologist or processor 900 may count the number of pops corresponding to the expansion of the at least one bellows 312 and relate that to the total number of bellow features to determine when the prime tube 300 is in the completely expanded configuration.

Referring now to FIG. 4E, a graph 400 illustrating fluid pressure within the prime tube 300 against time is shown for the embodiment of FIGS. 4A-4D. The fluid pressure, represented by curve 402, is initially substantially constant at section 404 as primarily air is injected into the internal chamber 350 and flows through the porous material of the closure 330. The fluid pressure then experiences several peaks 410 as medical fluid F enters the internal chamber 350 and causes each of the at least one bellows 316 to transition from the contracted state to the expanded state. In particular, because the medical fluid F cannot flow out of the distal end 314, the fluid pressure builds up until a first of the at least one bellows 316 transitions to the expanded state. As the first of the at least one bellows 316 expands, the fluid pressure drops due to the increase in volume of internal chamber 350 resulting from expansion of the bellows 316. As more fluid is expelled from the syringe 10A, 10B into the internal chamber 350, the pressure again builds until another of the bellows 316 expands. This process continues until all of the bellows 316 are in the expanded state and the priming operation is completed. After all bellows 316 are in the expanded state, the fluid pressure will then rise, as indicated by the tail section 412 of the curve 402, until the fluid flow is stopped.

In some embodiments, the priming status sensor 910 may include a motor current (or motor force) sensor configured to measure the current draw of the drive motor associated with the piston 13A, 13B. The controller 900 may be programmed or configured to detect changes in the fluid pressure based on changes in motor current. In particular, changes in motor current may correlate to changes in fluid pressure within the prime tube 300, as shown in the graph of FIG. 4E. When the motor current, and thus the fluid pressure continues to rise after the last bellows 316 is in the expanded state, the controller 900 may then stop the priming operation and indicate that the system is primed and can proceed to the next step in the injection protocol. Alternatively, after a predetermined number of bellows 316 have expanded, the controller 900 may determine that the system has been effectively primed.

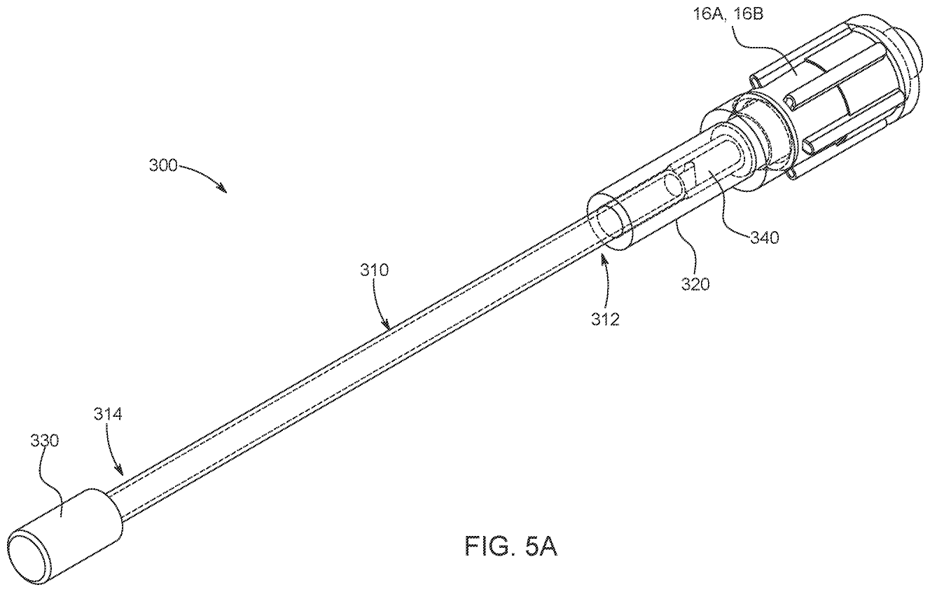

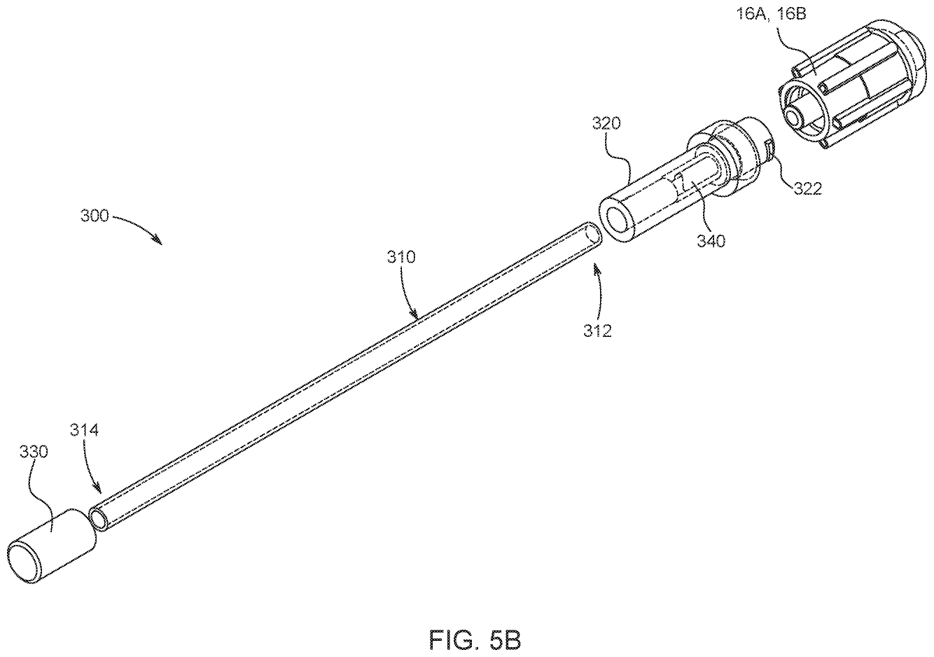

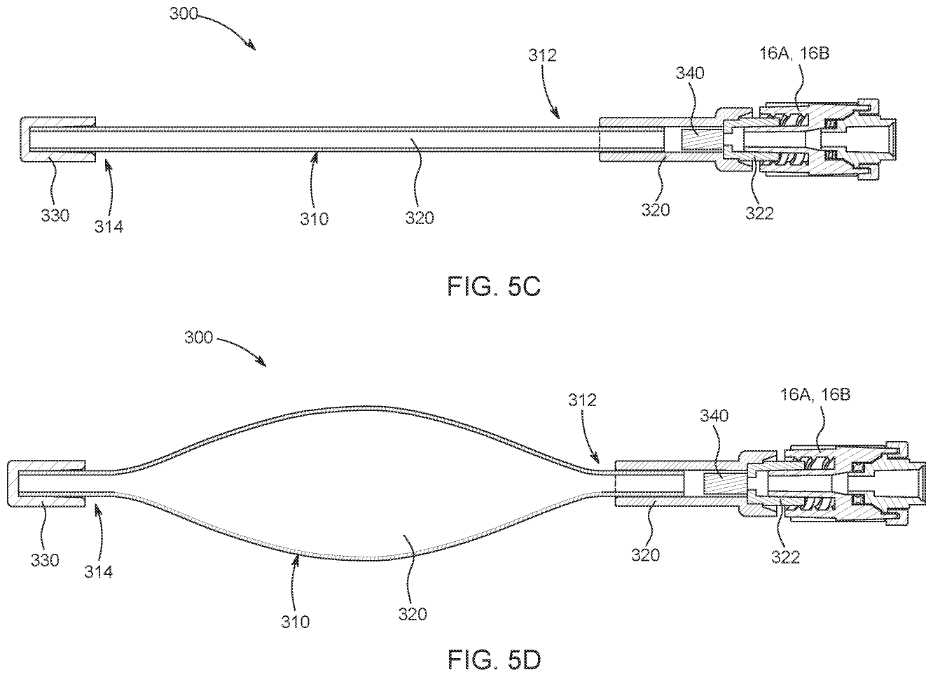

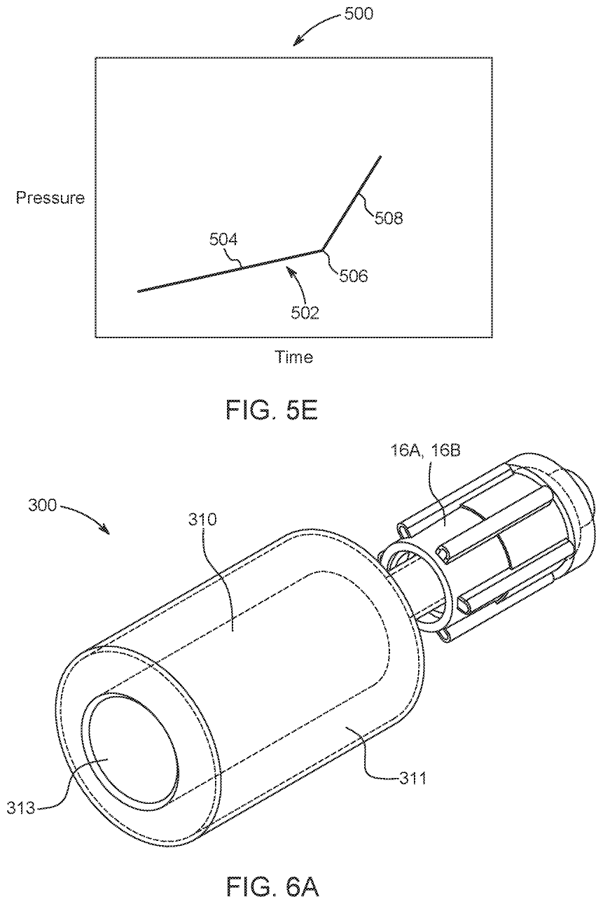

Referring now to FIGS. 5A-5D, an embodiment of a prime tube 300 including an expandable sidewall 310 is illustrated. The sidewall 310 may be made of a flexible or stretchable elastomeric material or balloon which expands when there is a pressure differential between the internal chamber 350 and the environment outside of the sidewall 310. The material of the sidewall 310 may be elastic in that when stretched, the sidewall 310 attempts to return to its initial state. Thus, if fluid pressure is removed from the internal chamber 350, the sidewall 310 will contract and force fluid back out of the proximal end 312 of the prime tube 300. The one-way valve 340 is provided to prevent backflow out of the proximal end 312 when the prime tube 300 is disconnected from the syringe. The prime tube 300 is provided in the non-expanded configuration shown in FIGS. 5A-5C.

With continued reference to FIGS. 5A-5D, the closure 330 in one embodiment may be a sealing plug inserted or otherwise connected to the distal end 314 of the prime tube 300. The sealing plug may prevent all fluid, including both air and medical fluid F, from exiting the distal end 314 of the prime tube 300. Due to the sealed nature of the sidewall 310 because of the one-way valve 340 and the closure 330, air and liquid is trapped in the internal chamber 350 during the priming operation. As the air is continued to be primed, eventually some medical fluid F pushes the remaining air into the prime tube 300 and the medical fluid F itself enters the internal chamber 350. As the volume of air and subsequently the fluid in the sidewall 310 increases, the pressure in the sidewall 310 increases and the flexible expandable walls of the sidewall 310 expand, similar to inflation of a balloon. In particular, as the pressure builds in the prime tube 300, the sidewall 310 begins to expand from the compressed state shown in FIGS. 5A-5C to the expanded state shown in FIG. 5D. Once the volume of the internal chamber 350 reaches a specific size, the technologist may determine that the syringe 10A, 10B has been primed and is ready for an injection procedure. In certain embodiments, the elastic characteristics of the sidewall 310 may be chosen so that it does not substantially expand under the pressurized air—due to the compressibility of gases—but expands when liquid pressure builds in the internal chamber 350.

In other embodiments, the closure 330 may be porous such that air can pass therethrough but it is impermeable to liquid. According to this embodiment and similar to the bellows (FIG. 4A-4E), the elastomeric sidewall 310 does not substantially expand when air flows into the internal chamber 350 since such air flows out through the closure 330. However, when liquid medical fluid F is injected into the internal chamber 350, the elastomeric sidewall 310 expands and the fluid pressure builds therein.

In some embodiments the priming status sensor 910 may include a camera, as described herein in connection with FIGS. 3 and 4A-4D, in conjunction with the controller 900 to determine that the syringe 10A, 10B has been primed based on the sidewall 310 being in the expanded state. As described herein in connection with FIGS. 4A-4D, the controller 900 may be programmed or configured to prevent performance of an injection procedure until a successful prime of the syringe 10A, 10B has been detected.

During removal of the prime tube 300 from the syringe 10A, 10B, the one-way valve 340 prevents the pressurized air and/or fluid in the internal chamber 350 from releasing from the proximal end 312 of the prime tube 300. The entire prime tube 300 with air and medical fluid F contained therein may be discarded according to hospital protocol.

Referring now to FIG. 5E, a graph 500 illustrating fluid pressure within the prime tube 300 against time is shown for the prime tube of FIGS. 5A-5D. The fluid pressure, represented by curve 502, initially rises slowly at section 504 as primarily air is injected into the internal chamber 350 with a low slope due to the compressibility of air. The fluid pressure continues to rise at a greater rate as medical fluid F pushes the remaining air out of the syringe 10A, 10B and some medical fluid F itself enters the internal chamber 350. The fluid pressure may experience a transition point 506 at which the sidewall 310 experiences a yield due to expansion of the sidewall 310. Once the total expanded volume of the internal chamber 350 is neared, the pressure increases at an even greater rate at section 508. In some embodiments, the priming status sensor 910 may include a motor current (or motor force) sensor configured to measure the current draw of the drive motor associated with the piston 13A, 13B. The controller 900 may be programmed or configured to detect changes in the fluid pressure based on changes in motor current and relate the pressure measurement to prime status. In particular, changes in motor current may correlate to changes in fluid pressure within the prime tube 300, as shown in the graph of FIG. 5E allowing controller 900 to determine when the system has been effectively primed.

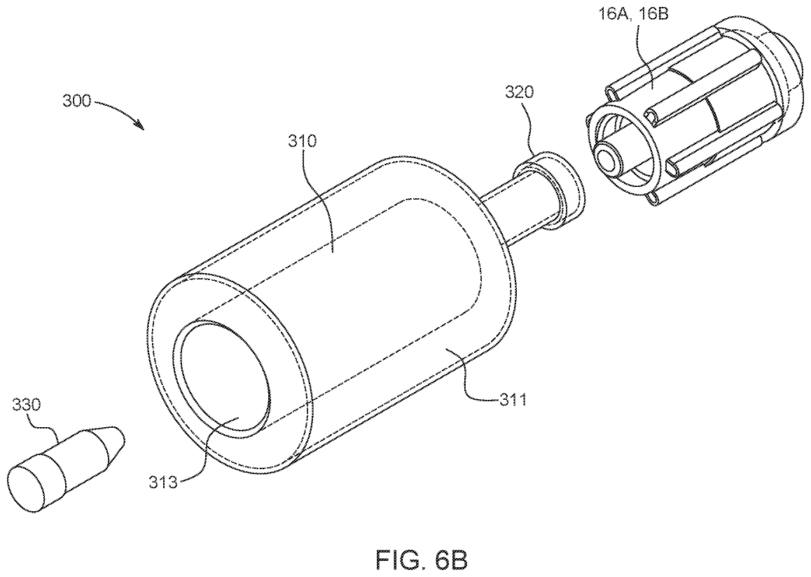

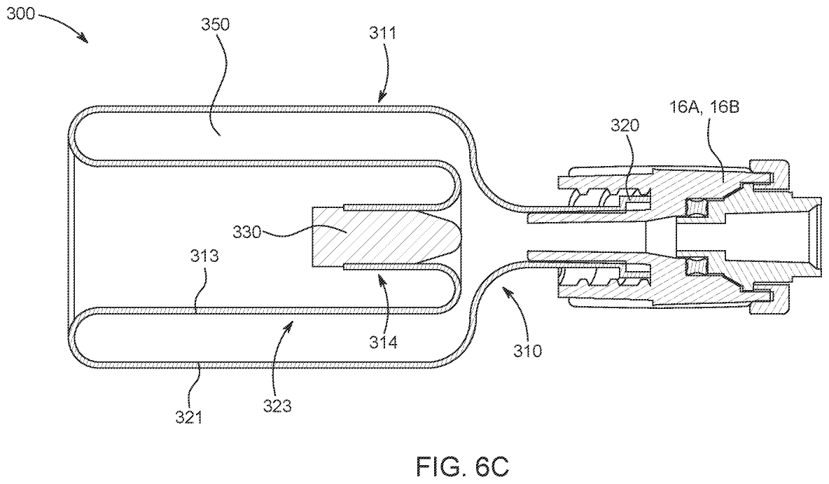

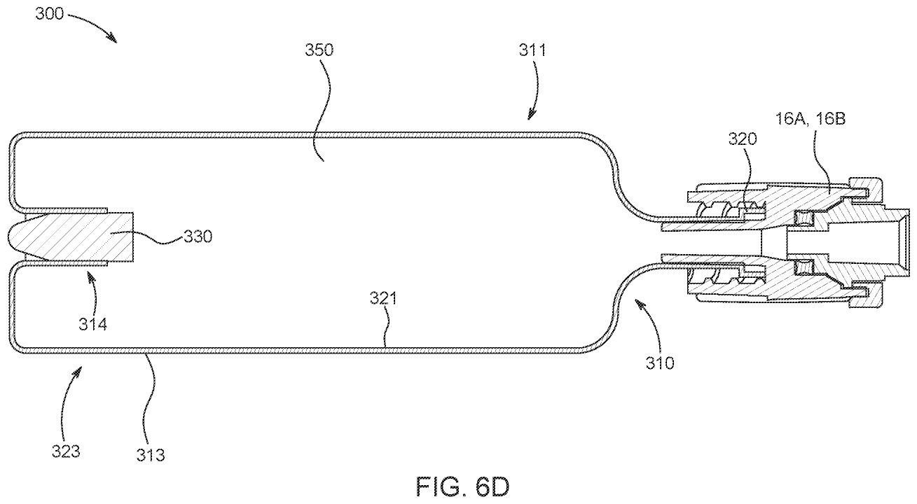

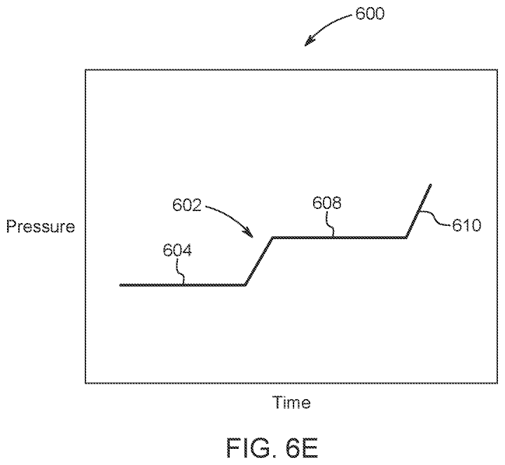

Referring now to FIGS. 6A-6D, an embodiment of a prime tube 300 is illustrated in which the sidewall 310 includes an expanding rolling diaphragm body. The sidewall 310 is flexible or resilient such that the sidewall 310 may be rolled or folded over itself in response to fluid pressure in the internal chamber 350. In the contracted state in which the prime tube 300 is initially provided, shown in FIGS. 6A-6C, the sidewall 310 is arranged such that a distal portion 313 of the sidewall 310 is rolled or folded into an interior space defined by a proximal portion 311 of the sidewall 310. The distal portion 313 is rolled over the proximal portion 311 such that an inner surface 323 of the distal portion 313 faces an inner surface 321 of the proximal portion 311. At least a portion of the distal portion 313 of the sidewall 310 is configured to invert in response to an increase in fluid pressure within the internal chamber 350 to transition the sidewall 310 to the expanded position shown in FIG. 6D. In particular, the distal portion 313 inverts by unrolling/unfolding from inside the proximal portion 311, such that the sidewall 310 assumes the expanded state shown in FIG. 6D. Consequently, the volume defined by the internal chamber 350 is greater with the sidewall 310 in the expanded state than in the contracted state.

With continued reference to FIGS. 6A-6D, the closure 330 in this embodiment may include a porous material substantially the same as the embodiment of FIGS. 4A-4D to allow air to flow through the closure 330 while retaining medical fluid F in the internal chamber 350. During a priming/purging operation of the syringe, air injected into the prime tube 300 from the syringe 10A, 10B passes through the internal chamber 350 and exits through the porous material of the closure 330 at the distal end 314. Due to the compressibility of air, the rolling diaphragm portion of the sidewall 310 remains substantially unchanged, i.e., remains in the contracted state, as air is ejected from the syringe 10A, 10B. As the air is continued to be primed, the medical fluid F pushes the remaining air from the syringe 10A, 10B into the prime tube 300 and out the porous material of the closure 330, and the medical fluid F itself enters the internal chamber 350. Due to the hydrophobic nature of the closure 330, the medical fluid F is unable to exit the internal chamber 350. As the volume of the medical fluid F in the internal chamber 350 increases, the pressure in the internal chamber 350 increases, forcing the distal portion 313 to unroll/unfold from the interior of the proximal portion 311, thus transitioning the sidewall 310 from the contracted state shown in FIGS. 6A-6C to the expanded state shown in FIG. 6D.

Once the volume of the internal chamber 350 reaches a specific size or the rolling diaphragm portion of the sidewall 310 reaches a specific unrolled state, e.g., completely unrolled, the technologist may determine that substantially all air has been purged from the syringe 10A, 10B, meaning the syringe 10A, 10B is primed and ready for use. In some embodiments, the priming status sensor 910 (see FIGS. 2B and 3) may include a detector for image recognition, such as a camera as described in U.S. Pat. No. 10,201,666. Based on an output signal from the priming status sensor 910, the controller 900 may be programmed or configured determine that the prime tube 300 is in the expanded state and the syringe 10A, 10B is primed of air. The processor 900 may be configured to prevent a fluid injection procedure until it receives indication that the syringe 10A, 10B has been successfully primed. Once a successful prime has been established as determined by the controller 900 and/or technologist, the prime tube 300 may be removed and discarded according to hospital protocol, and a suitably primed patient catheter line may be installed on the syringe 10A, 10B.

Referring now to FIG. 6E, a graph 600 illustrating fluid pressure within the prime tube 300 against time is shown for the embodiment of FIGS. 6A-6D. The fluid pressure, represented by curve 602, is initially substantially constant at section 604 as primarily air is injected into the internal chamber 350 and flows through the porous material of the closure 330. The fluid pressure then increases as medical fluid F enters the internal chamber 350, until the fluid pressure reaches a sufficient pressure to cause the distal portion 313 of the sidewall 310 to unroll from the proximal portion 311. The fluid pressure then continues to remain substantially constant at section 608 as more fluid is expelled from the syringe 10A, 10B into the internal chamber 350, and continues to cause the sidewall 310 to unroll towards the expanded state. After the sidewall 310 is in the substantially unrolled state the volume of the internal chamber 350 is substantially maximized, the fluid pressure rises substantially at section 610 with continued ejection of fluid from the syringe.

In some embodiments, the priming status sensor 910 may include a motor current (or motor force) sensor configured to measure the current draw of the drive motor associated with the piston 13A, 13B. The controller 900 may be programmed or configured to detect changes in the fluid pressure based on changes in motor current. In particular, changes in motor current may correlate to changes in fluid pressure within the prime tube 300, as shown in the graph of FIG. 6E.

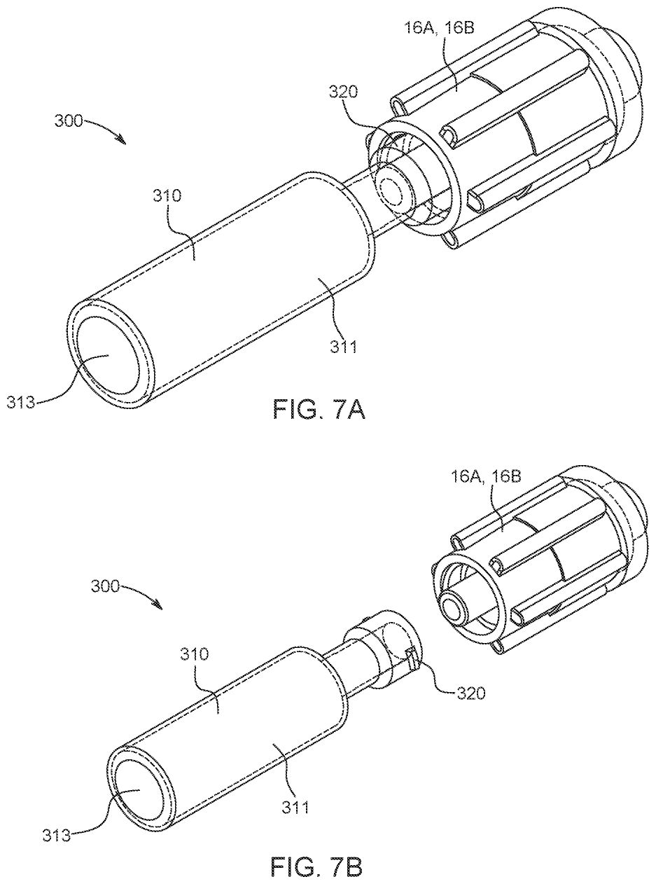

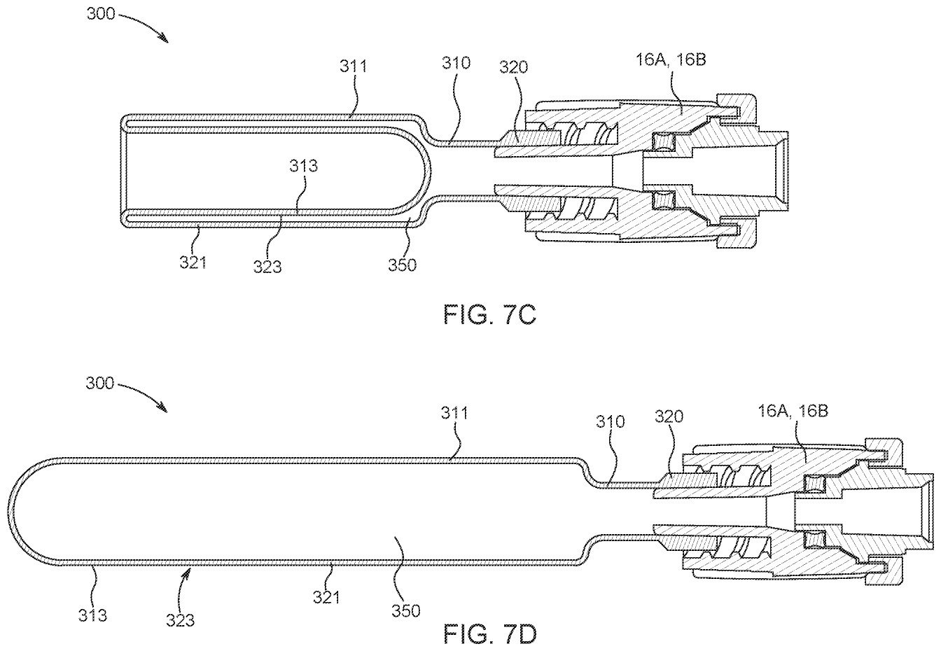

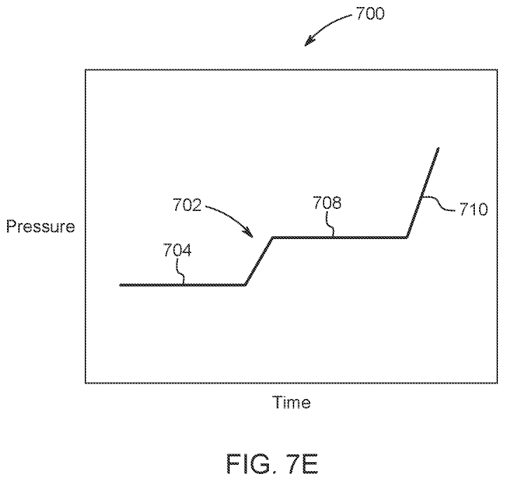

Referring now to FIGS. 7A-7D, an embodiment of a prime tube 300 is illustrated which includes many of the same features, such as the rolling diaphragm sidewall 310, of the embodiment of FIGS. 6A-6D. The primary difference between these embodiments is that the distal end 314 of the sidewall 310 is completely sealed in the embodiment of FIGS. 7A-7D, such that air cannot exit the distal end 314. As a result, all fluid, including both air and medical fluid F, ejected from the syringe 10A, 10B during the priming/purging operation is retained within the internal chamber 350. Due to the compressibility of air and non-compressibility of the medical fluid, the rolling diaphragm will remain substantially in the rolled state as air is ejected into prime tube 300 but then transition to the expanded state as fluid is ejected into the internal chamber 350 and the fluid pressure within the internal chamber 350 builds. Prime states may then be recognized by wither the technologist or by the controller 900 using input from a priming status sensor 910, as described herein.

Referring now to FIG. 7E, a graph 700 illustrating fluid pressure within the prime tube 300 against time is shown for the embodiment of FIGS. 7A-7D. The fluid pressure, represented by curve 702, is initially substantially constant at section 604 as primarily air is injected into the internal chamber 350 and flows through the porous material of the closure 330. The fluid pressure then increases as medical fluid F enters the internal chamber 350, until the fluid pressure reaches a sufficient pressure to cause the distal portion 313 of the sidewall 310 to unroll from the proximal portion 311. The fluid pressure then continues to remain substantially constant at section 708 as more fluid is expelled from the syringe 10A, 10B into the internal chamber 350, and continues to cause the sidewall 310 to unroll towards the expanded state. After the sidewall 310 is in the substantially unrolled state the volume of the internal chamber 350 is substantially maximized, the air/fluid pressure rises substantially at section 710 with continued ejection of fluid from the syringe 10A, 10B. The pressure behavior of the embodiment of FIGS. 7A-7D is thus similar to the embodiment of FIGS. 6A-6D, although the presence of the porous closure 330 in the embodiment of FIGS. 6A-6D may at least initially reduce pressure within the internal chamber 350 as air is able to escape.

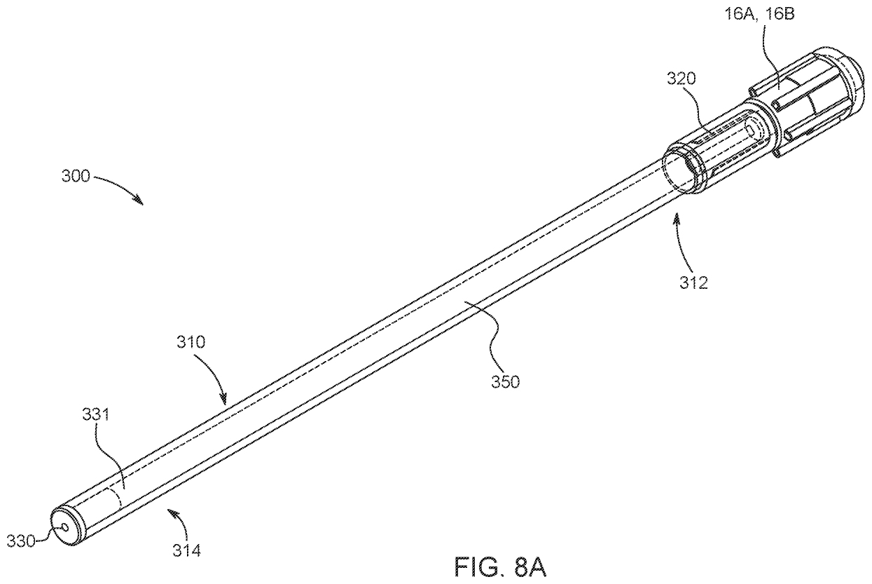

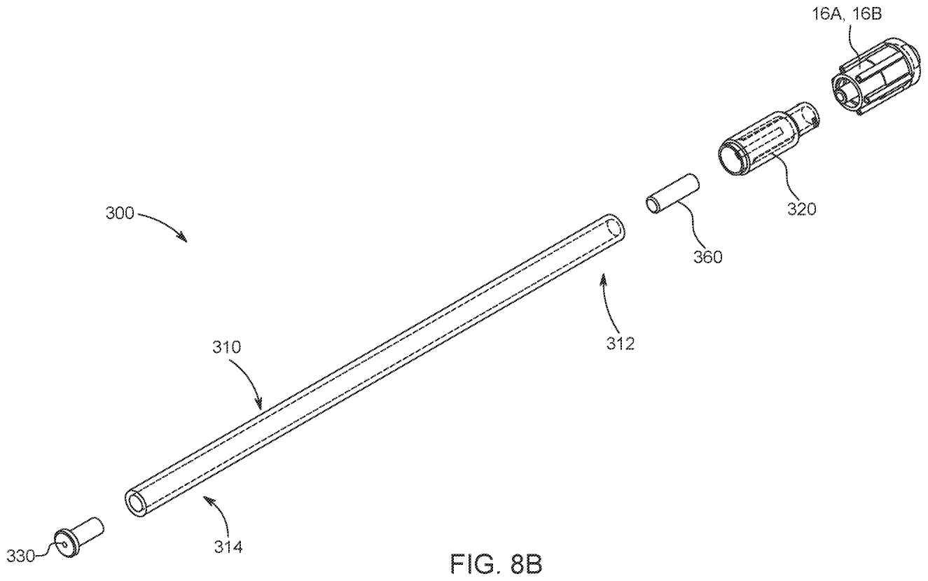

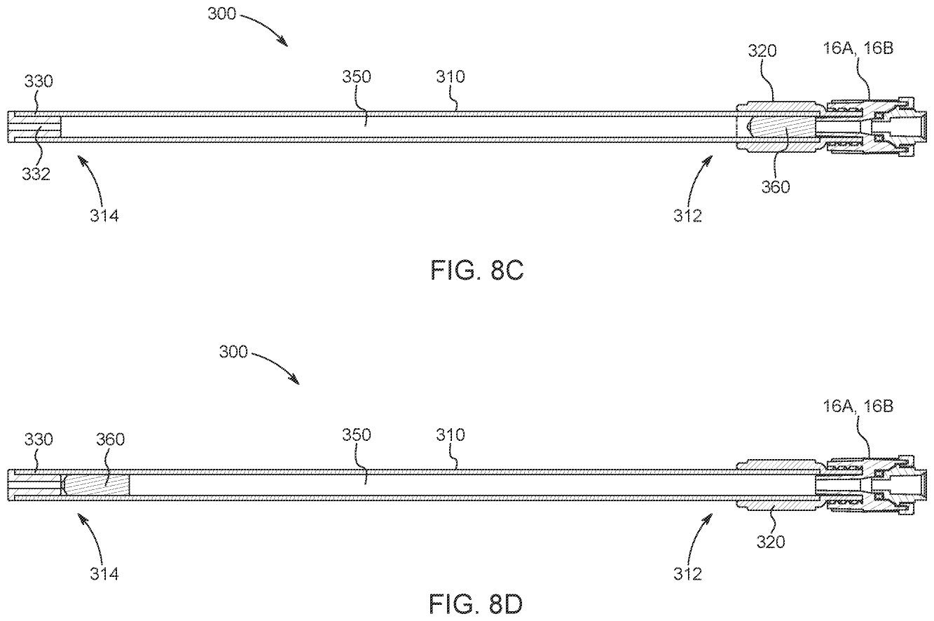

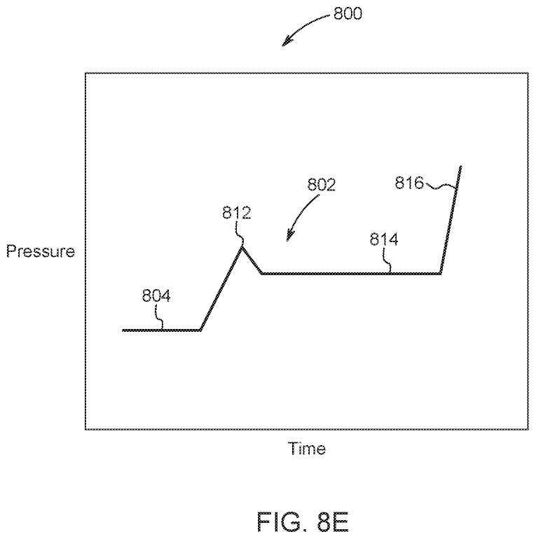

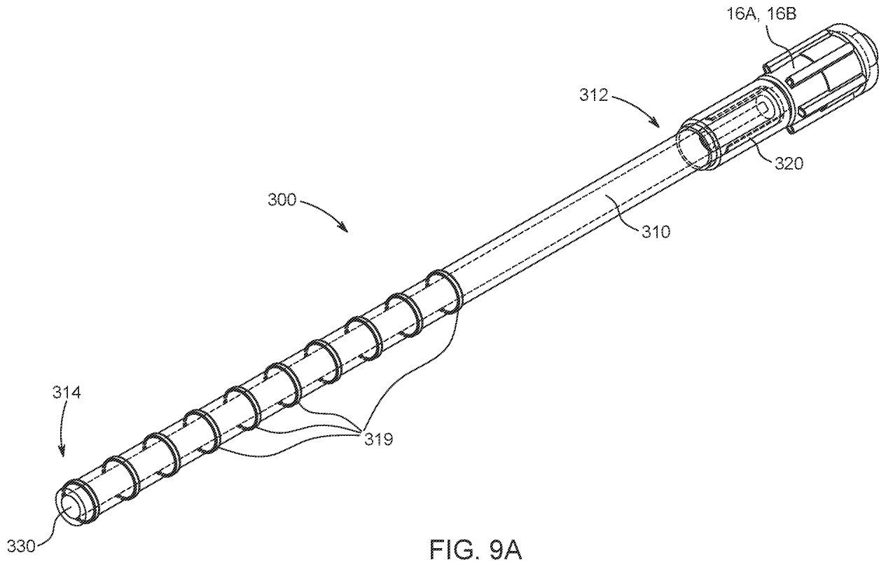

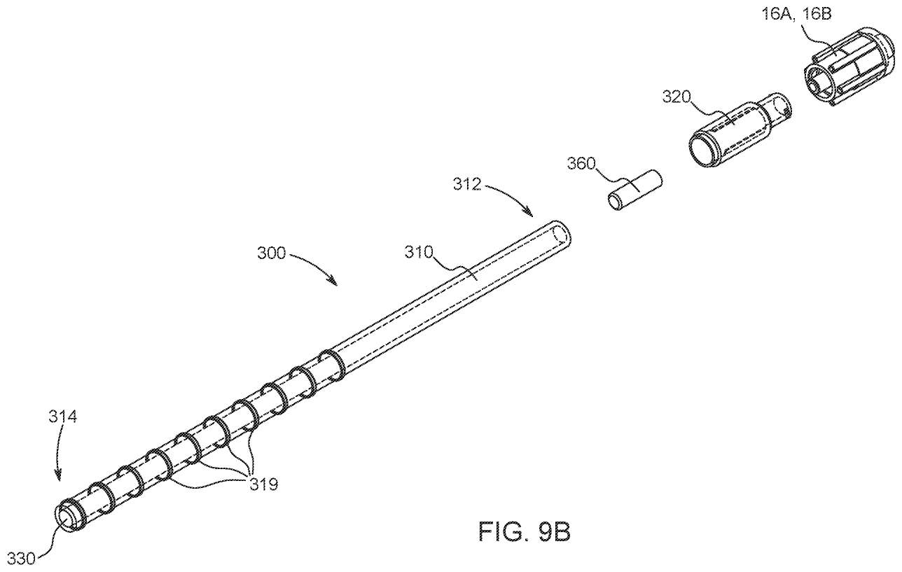

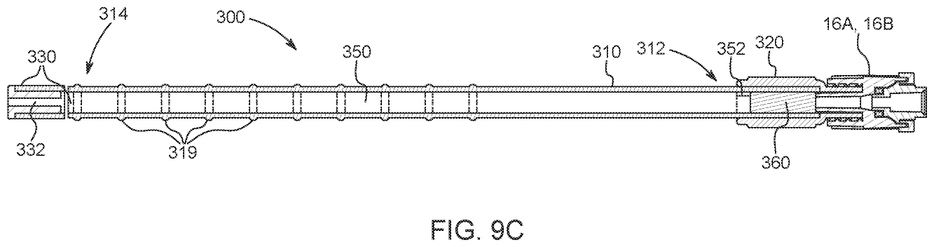

Referring to FIGS. 8A-9C, embodiments of a prime tube 300 including a shuttle member 360 slidable within the internal chamber 530 is illustrated. According to these embodiments, the sidewall 310 may be substantially rigid and non-elastic, such that under normal priming pressures below a predetermined threshold the sidewall 310 does not appreciably expand. The shuttle member 360 is configured to slide within the internal chamber 350 from the proximal end 312 toward the distal end 314 in response to liquid being ejected into the prime tube 300 from the syringe 10A, 10B. The prime tube 300 is initially provided with the shuttle member 360 adjacent to or near the proximal end 312. In some embodiments, the shuttle member 360 may be sized to have a frictional fit with the sidewall 310 such that the shuttle member 360 cannot be inadvertently dislodged from the proximal end 312 until the prime tube 300 is pressurized from the syringe 10A, 10B. In some embodiments, the proximal end 312 of the sidewall 310 has a slightly reduced diameter to increase the frictional fit with the shuttle member 360 at the proximal end 312. As such, the initial amount of fluid pressure required to dislodge the shuttle member 360 is greater than the fluid pressure required to move the shuttle member 360 the remainder of the distance to the distal end 314. In some embodiments, as shown in FIG. 9C, the sidewall 310 may include an internal lip 352 to retain the shuttle member 360 adjacent to or near the proximal end 312 until the prime tube 300 is pressurized from the syringe 10A, 10B. The lip 352 or friction fit may be such that the shuttle member 360 is not dislodged under air pressure but is dislodged and becomes slidable when fluid flows into the internal chamber 350.

In some embodiments, the shuttle member 360 may allow passage of air to the distal end 314 of the prime tube 300. In such embodiments, the shuttle member 360 may be made of a hydrophobic medical grade plastic, as described herein, that are permeable to air but impermeable to the medical fluid F. In some embodiments, the dimensional tolerance between the outside of the shuttle member 360 and an the inside of the sidewall 310 may be such that air can pass between the shuttle member 360 and the sidewall 310, but medical fluid F cannot substantially pass between the shuttle member 360 and the sidewall 310. In some embodiments, the shuttle member 360 may include one or more apertures having sufficient cross-sectional area to allow the passage of air but substantially prevent the passage of medical fluid F.

The closure 330 is adhered to an aperture 331 at the distal end 314 of the prime tube 300 and may act as a stop to prevent the shuttle member 360 from being discharged out the distal end 314 of the prime tube 300. The closure 330 may include an aperture 332 to allow air to exit the distal end 314 of the prime tube 300. Various embodiments may include a lip or protrusion at the distal end 314 of the sidewall 310 instead of a closure 330, where the inner diameter of the distal end 314 is less than the inner diameter of the sidewall 310 to prevent the shuttle member 360 from being discharged out the distal end 314 of the prime tube 300.

During a priming/purging operation of the syringe 10A, 10B, air passes through or around the shuttle member 360, through the internal chamber 350, and out the aperture 332 of the closure 330. In some embodiments, air may pass through or around the shuttle member 360 without substantially dislodging and/or moving the shuttle member 350 within the internal chamber 350. As the air is continued to be primed, eventually some medical fluid F pushes the remaining air from the syringe 10A, 10B into the internal chamber 350, and the medical fluid F itself enters the internal chamber 350. The pressurized medical fluid F dislodges the shuttle member 360 from the frictional fit with the sidewall 310 and causes the shuttle member 360 to slide distally within the internal chamber 350 toward the closure 330. As medical fluid F continues to be injected into the internal chamber 350 from the syringe 10A, 10B, the shuttle member 360 may continue sliding distally until the shuttle member 360 contacts the closure 330 and substantially all of the air has been forced out of the internal chamber 350 by the pressurized medical fluid F.

With continued reference to FIGS. 8A-9C, the sidewall 310 may be made of a translucent or transparent material such that the shuttle member 360 is visible through the sidewall 310. The shuttle member 360 may be a readily visualized, conspicuous color, such that the movement of the shuttle member 360 along the longitudinal axis of internal chamber 350 may be observed or detected. Once a predetermined volume of the medical fluid F has entered the prime tube 300, as evidenced by the longitudinal position of the shuttle member 360 within the internal chamber 350, the technologist may determine that the syringe 10A, 10B has been fully primed. In some embodiments, the internal chamber 350 may be sized such that the shuttle member 360 contacts the closure 330 (as shown in FIG. 8D) once a volume of medical fluid F sufficient to prime the syringe 10A, 10B has been ejected into the internal chamber 350. In such embodiments, a ceasing of movement of the shuttle member 360, indicating that the shuttle member 360 has engaged the closure 330, may be observed by the technologist to establish that the syringe 10A, 10B has been fully primed.

In some embodiments, the priming status sensor 910 (see FIGS. 2B and 3) may be configured to detect the position of the shuttle member 360 within the internal chamber 350. Referring specifically to FIGS. 9A-9C, the sidewall 310 may include or more ribs 319 or other demarcations or indicators that the technologist and/or the priming status sensor 910 may use as a scale to assist in determining relative position and distance travelled of the shuttle member 360 within the internal chamber 350 and thus, the volume of medical fluid F that has entered the prime tube 300. The priming status sensor 910 may include a detector for image recognition, such as a camera as described in U.S. Pat. No. 10,201,666. Based on an output signal from the priming status sensor 910, the controller 900 may be programmed or configured to determine that the shuttle member 360 has moved a sufficient distance indicating that the syringe 10A, 10B is primed of air. The processor 900 may be configured to prevent a fluid injection procedure until it receives indication that the syringe 10A, 10B has been successfully primed. Once a successful prime has been established as determined by the controller 900 and/or technologist, the prime tube 300 may be removed and discarded according to hospital protocol, and a suitably primed patient catheter line may be installed on the syringe 10A, 10B. The prime tube 300 may include a one-way check valve at the proximal end to prevent fluid leakage upon removal.

In some embodiments, the aperture 332 of the closure 330 may be sized or have flow features configured to emit an audible sound, such as a whistle, when air flows through the aperture 332. The technologist may determine that the syringe 10A, 10B has been primed when the aperture 332 ceases to emit a sound, indicating that substantially all of the air has been expelled from the internal chamber 350. Alternatively, the priming status sensor 910 may include a microphone configured to detect the sound emitted from the aperture 332. The controller 900 may be programmed or configured to determine that the syringe 10A, 10B has been primed when the aperture 332 ceases to emit a sound, indicating that substantially all of the air has been expelled from the internal chamber 350.

Referring now to FIG. 8E, a graph 800 illustrating fluid pressure within the prime tube 300 against time is shown for the embodiment of FIGS. 8A-8D. The fluid pressure, represented by curve 802, is initially substantially constant at section 804 as primarily air is injected into the internal chamber 350 and flows through or around the shuttle member 360. The fluid pressure then increases as medical fluid F enters the internal chamber 350, until the fluid pressure reaches a sufficient pressure to dislodge the shuttle member 360 from the proximal end 314. Dislodgement of the shuttle member 360 may cause a spike 812 in pressure as the initial static friction between the shuttle member 360 and the sidewall 310 is overcome. The pressure may then stabilize at section 814 as the shuttle member 360 is displaced by the medical fluid F and slides along the prime tube 300 until it reaches the distal end 314. Once the shuttle member 360 contacts the distal end, fluid pressure builds quickly in the internal chamber 350, as indicated by section 816.

In some embodiments, the priming status sensor 910 may include a motor current (or motor force) sensor configured to measure the current draw of the drive motor associated with the piston 13A, 13B. The controller 900 may be programmed or configured to detect changes in the fluid pressure based on changes in motor current. In particular, changes in motor current may correlate to changes in fluid pressure within the prime tube 300, as shown in the graph of FIG. 8E. For example, when the pressure stabilizes at section 814 or rises dramatically at section 816, the controller 900 may determine that the air has been primed from the syringes and provide an indication that the priming operation is complete

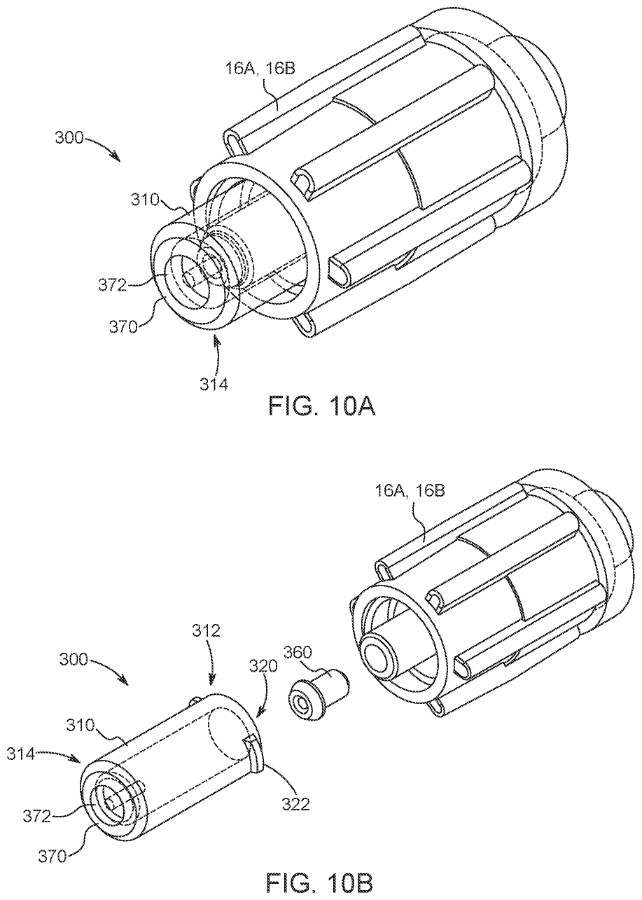

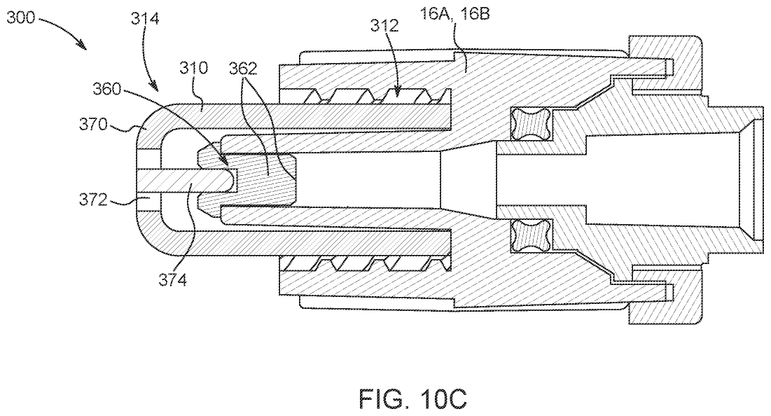

Referring now to FIGS. 10A-10C, an embodiment of a prime tube 300 is shown in which the sidewall 310 defines a cap 370 at the distal end 314 having one or more air release holes 372 and a plug retention member 374. A shuttle member 360 in the form of a porous plug is disposed within the cap 370, and in an initial state or position of the prime tube 300 is held in the outlet 16A, 16B of the syringe 10A, 10B by the plug retention member 374. In some embodiments, the shuttle member 360 may be made from a hydrophobic medical grade plastic, as described herein, that are permeable to air but impermeable to the medical fluid F. In some embodiments, the shuttle member 360 may be a solid material defining one or more apertures having a sufficient cross-sectional area to allow the passage of air but an insufficient cross-sectional area to allow the passage of the medical fluid F.

During a priming/purging operation, air ejected from the syringe 10A, 10B flows through the porous shuttle member 360, into the internal chamber 350, and out the one or more air release holes 372. As the air is continued to be primed and purged out of the syringe 10A, 10B, eventually some medical fluid F pushes the remaining air from the syringe 10A, 10B through the shuttle member 360 and the medical fluid F contacts a proximal face 362 of the shuttle member 360. As the medical fluid F is unable to pass through the shuttle member 360, fluid pressure builds against the proximal face 362 causing the shuttle member 360 to move to a second, primed position and bear against the plug retention member 374.

In some embodiments, the controller 900 may be configured to measure the fluid pressure at the proximal face 362 of the shuttle member 360. In particular, the priming status sensor 910 may include a motor current (or motor force) sensor configured to measure the current draw of the drive motor associated with the piston 13A, 13B. The controller 900 may be programmed or configured to determine the fluid pressure based on motor current draw. The controller 900 may be programmed or configured to determine that the syringe 10A, 10B has been primed when a predetermined fluid pressure is measured. Once a successful prime has been established by the controller 900, the prime tube 300 may be removed and discarded according to hospital protocol, and a suitably primed patient catheter line may be installed on the syringe 10A, 10B. One feature of the prime tube 300 illustrated in FIGS. 10A-10C is that the volume of medical fluid F ejected during the priming operation is minimized, reducing hospital waste and saving on costs. Once the controller 900 has determined that the syringe has been primed, the controller may release the pressure on the piston 13A, 13B and depressurize the fluid in the syringe to ensure that pressurized fluid is not ejected from the syringe when the prime tube 300 is removed.

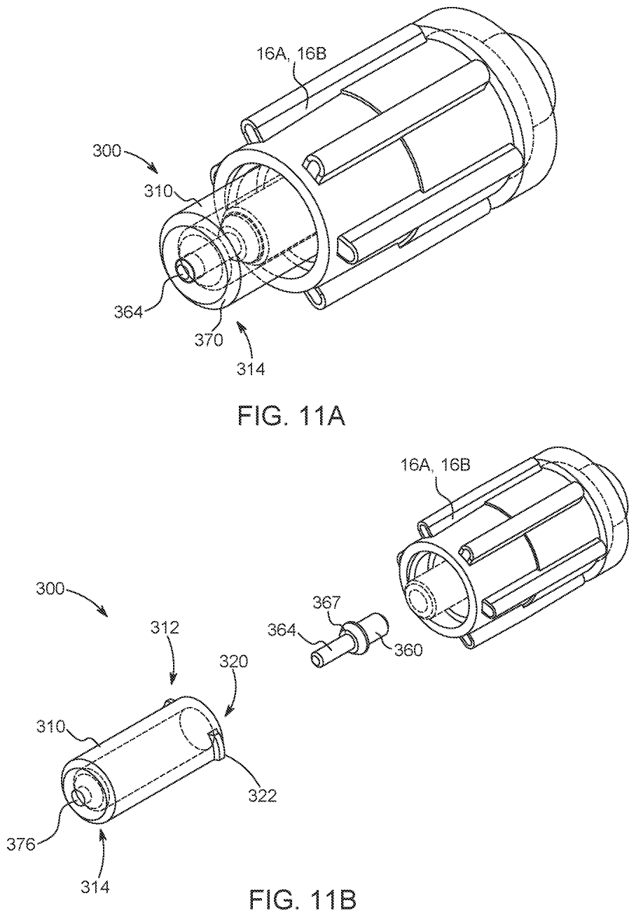

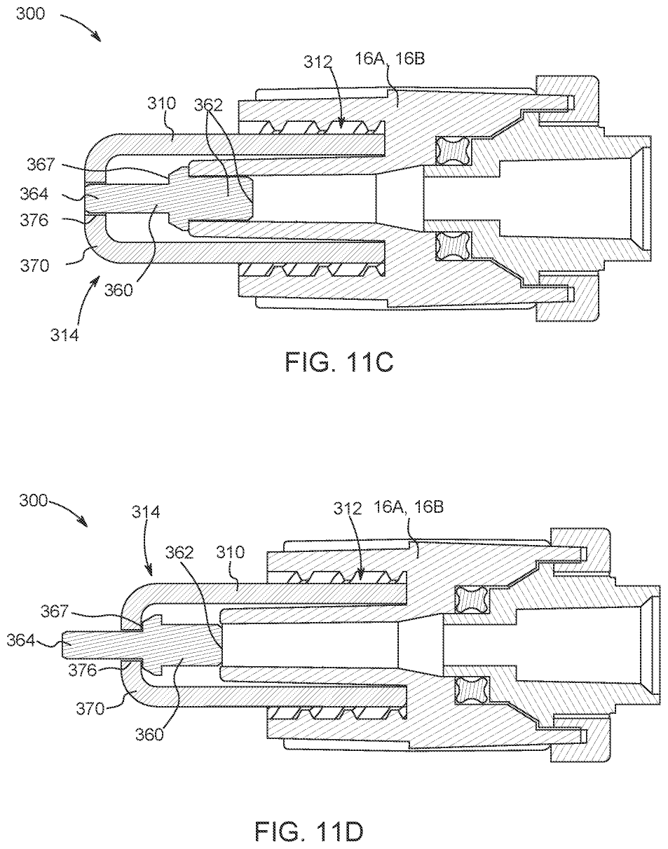

Referring now to FIGS. 11A-11D, an embodiment of a prime tube 300 having a distally extending indicator tab is illustrated. The sidewall 310 defines a cap 370 at the distal end 314 having a receiving aperture 376. The sidewall 310 engages with a threaded connector of an outlet 16A, 16B of the syringe 10A, 10B by a threadable lug 320. A shuttle member 360 in the form of a porous plug is disposed within the internal chamber 350, and in an initial state or position of the prime tube 300 the shuttle member 360 is held in the outlet 16A, 16B of the syringe 10A, 10B by a press fit. In some embodiments, the shuttle member 360 may be made from a hydrophobic medical grade plastic, as described herein, that are permeable to air but impermeable to the medical fluid F. In some embodiments, the shuttle member 360 may be a solid material defining one or more apertures having a sufficient cross-sectional area to allow the passage of air but an insufficient cross-sectional area to allow the passage of the medical fluid F. In certain embodiments, the shuttle member 360 may be colored a conspicuous color to be readily viewed by the technologist. The shuttle member 360 includes a distally extending tip 364 axially aligned with the receiving aperture 376 of the cap 370. The diameter of the receiving aperture 376 may be slightly larger than the diameter of the distally extending tip 364 to allow air to pass between the space between the diameters.

During a priming/purging operation, air ejected from the syringe 10A, 10B flows through the porous shuttle member 360, into the internal chamber 350, and out the receiving aperture 376. As the air is continued to be primed and purged out of the syringe 10A, 10B, eventually some medical fluid F pushes the remaining air from the syringe 10A, 10B through the shuttle member 360, and the medical fluid F contacts the proximal face 362 of the shuttle member 360. As the medical fluid F is unable to pass through the shuttle member 360, fluid pressure builds against the proximal face 362 causing the shuttle member 360 to be dislodged from the outlet 16A, 16B of the syringe 10A, 10B to a second, primed position and bear against the cap 370. The tip 364 extends through the receiving aperture 376, providing an indication to the technologist and/or the controller 900 that the shuttle member 360 has been dislodged and the syringe 10A, 10B has been primed. A distal surface 367 of the shuttle member 360 may sealably abut an inner surface surrounding the receiving aperture 376 to prevent any medical fluid from escaping from the prime tube 300 once it has been primed.

In some embodiments, the priming status sensor 910 may include a detector for image recognition, such as a camera as described in U.S. Pat. No. 10,201,666. Priming status sensor 910 may be configured to detect movement and/or position of the tip 364 from an initial position in which tip 364 is flush with or recessed in the sidewall 310 (see FIG. 11C) to a primed position (see FIG. 11D) in which shuttle member 360 moves to the distal end 314 of the prime tube 300 and tip 364 extends distally through the receiving aperture 376. In some embodiments, priming status sensor 910 may include a limit switch contacted by the tip 364 when shuttle member 360 moves to the distal end 314 of prime tube 300.

Based on an output signal from the priming status sensor 910, the controller 900 may be programmed or configured to determine that the shuttle member 360 has moved to the distal end 314 of the prime tube 300, indicating that the syringe 10A, 10B has been primed. The processor 900 may be configured to prevent a fluid injection procedure until it receives indication that the syringe 10A, 10B has been successfully primed. Once a successful prime has been established as determined by the controller 900 and/or technologist, the prime tube 300 may be removed and discarded according to hospital protocol, and a suitably primed patient catheter line may be installed on the syringe 10A, 10B.

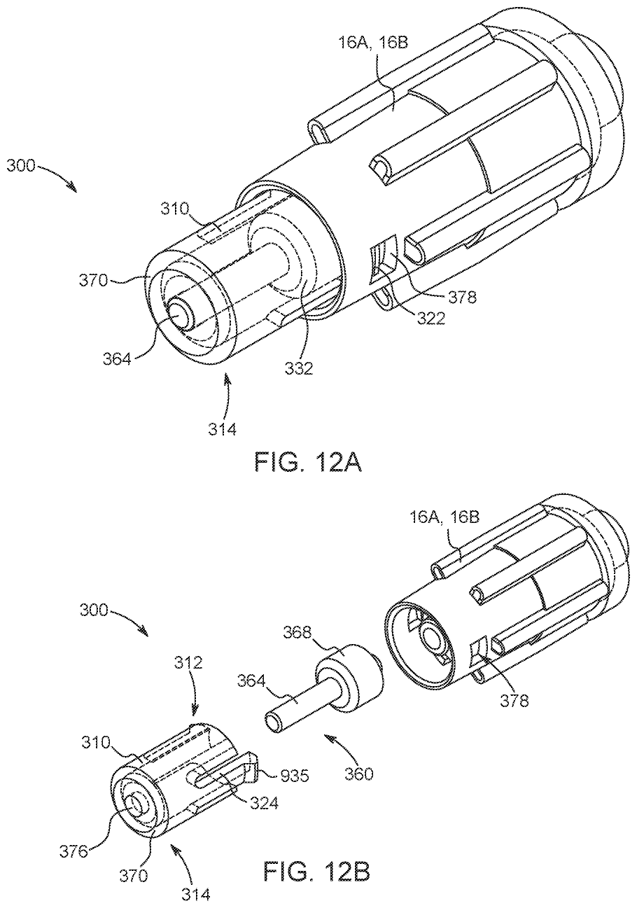

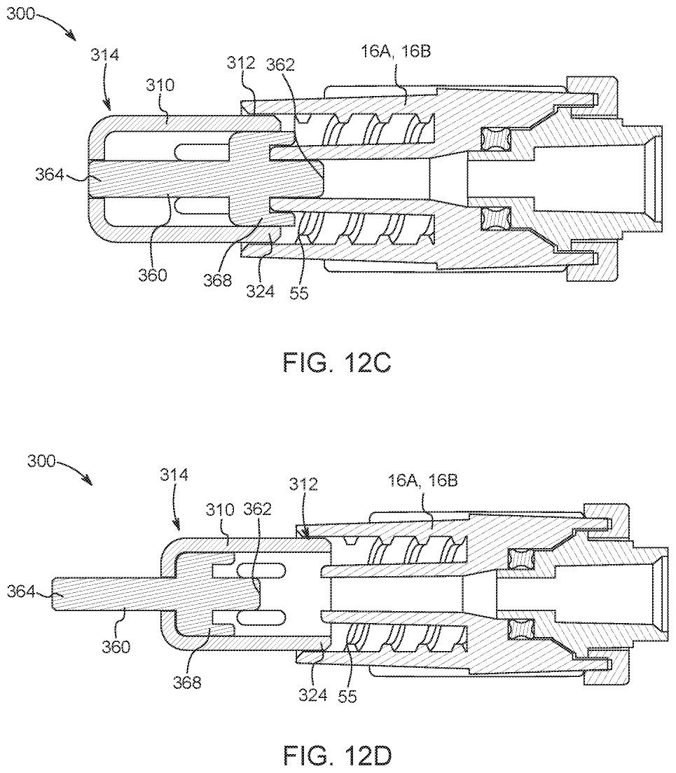

Referring now to FIGS. 12A-12C, an embodiment of a prime tube 300 is similar to the embodiment of FIGS. 11A-11D, and the primary differences will be discussed. In the embodiment shown in FIGS. 12A-12C, the engaging lugs 322 are provided on deflectable arms 324 of the connector 320. The engaging lugs 322 may, in some embodiments, include tabs that fit into corresponding slots 378 in the outlet 16A, 16B of the syringe 10A, 10B. The proximal end of the shuttle member 360 includes an at least partially circumferential wall 368 that surrounds an outer circumference of the outlet 16A, 16B and engages the deflectable arms 324 in the initial position of the prime tube 300 (shown in FIG. 12C) to prevent inward deflection of the arms 324. Engagement of the circumferential wall 368 with the deflectable arms 324 reversibly locks the engaging lugs 322 of the prime tube 300 in the corresponding slots 378 the outlet 16A, 16B of the syringe 10A, 10B, and prevents manual removal of the connector 320 of the prime tube 300 from the outlet 16A, 16B of the syringe 10A, 10B when the shuttle member 360 is sealed in the outlet 16A, 16B. As before, the shuttle member 360 is permeable to air and impermeable to liquid, so as fluid pressure builds, the shuttle member 360 is dislodged from the outlet 16A, 16B. When the shuttle member 360 moves distally to the primed position, the circumferential wall 368 disengages from between the outer circumference of the outlet 16A, 16B and the deflectable arms 324 (as shown in FIG. 12D), allowing the arms 324 to deflect radially inward such that the engagement lugs 322 can release from the slots 378. The prime tube 300 can then be removed from the syringe 10A, 10B. Thus, the arrangement of the circumferential wall 368 and the deflectable arms 324 forces the technologist to perform a priming operation on the syringe 10A, 10B before the prime tube 300 can be removed from the syringe 10A, 10B ensuring that the syringes are primed before the injection procedure can proceed (i.e., by removal of the prime tubes 300). As before, the technologist and/or processor 900 can determine the status of the system (primed or unprimed) by the tip 364 extending through receiving aperture 376 and by the technologist being able to remove prime tube 300.

In some embodiments, the engaging lugs 322 of the connector 320 may be configured to automatically release from the slots 378 of the outlet 16A, 16B of the syringe 10A, 10B at a predetermined fluid pressure corresponding to a fluid pressure at which the syringe 10A, 10B is fully primed. Thus, release of the prime tube 300 from the syringe 10A, 10B indicates that the syringe 10A, 10B is primed and ready for an injection protocol. In some embodiments, the connector 320 may be configured so as to not be manually removable from the outlet 16A, 16B of the syringe 10A, 10B, thereby forcing the technologist to perform a priming operation to automatically release the prime tube 300 from the syringe 10A, 10B. The syringe 10A, 10B may be oriented such that the prime tube 300 falls into a waste container when the engaging lugs 322 release from the outlet 16A, 16B of the syringe 10A, 10B. A suitably primed patient catheter line may be installed on the syringe 10A, 10B after the prime tube 300 has been released.

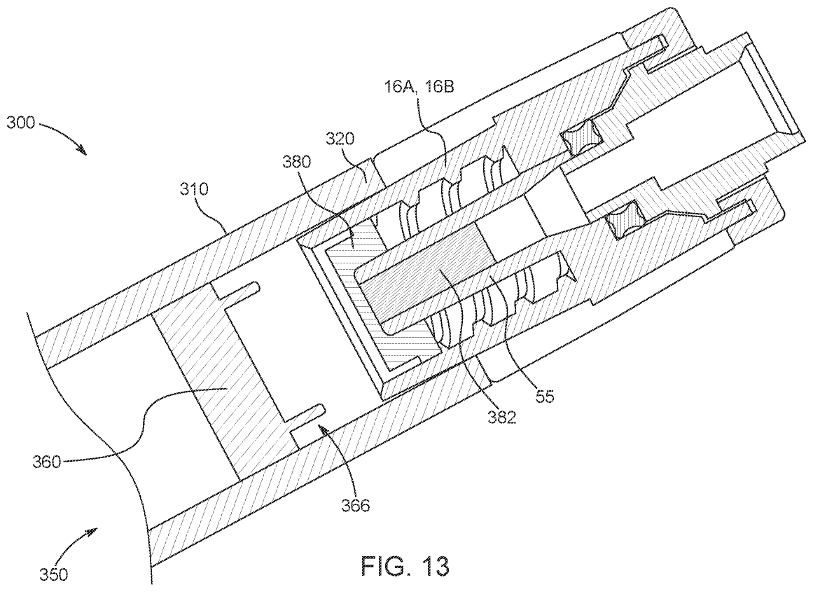

Referring now to FIG. 13, an embodiment of a prime tube 300 including an inner cap 380 that fits over the inner nozzle 55 of the outlet 16A, 16B of the syringe 10A, 10B is illustrated. The cap 380 is configured and/or arranged to be recessed into the outlet 16A, 16B so that the cap 380 cannot be manually removed from the inner nozzle 55 prior to the syringe being primed and can only be removed by a priming operation. Thus, an injection procedure cannot be performed without initial priming and resultant removal of the cap 380. For example, the inner cap 380 may be recessed by a friction fit within the outlet 16A, 16B so that a person cannot deliberately or inadvertently remove the inner cap 380 with ordinary effort. In some embodiments, the inner cap 380 may include a proximal post 382 that inserts into at least a portion of the inner nozzle 55 of the syringe 10A, 10B. The proximal post 382 may help maintain the inner cap 380 in place on the inner nozzle 55 via a press fit or friction fit. In some embodiments, the inner cap 380 may be made from a hydrophobic medical grade plastic, as described herein, that is permeable to air but impermeable to the medical fluid F. In some embodiments, the inner cap 380 may be a solid material defining one or more apertures having a sufficient cross-sectional area to allow the passage of air but an insufficient cross-sectional area to allow the passage of the medical fluid F.

According to certain embodiments, a shuttle member 360 may be disposed and slidable within the interior chamber 350 of prime tube 300 and has a sufficiently tight fit to the sidewall 310 to prevent medical fluid F from flowing between the shuttle member 360 and the sidewall 310. In some embodiments, the shuttle member 360 may include fingers 366 configured to engage the cap 380. In some embodiments, the shuttle member 360 may be made from a hydrophobic medical grade plastic, as described herein, that is permeable to air but impermeable to the medical fluid F. In some embodiments, the shuttle member 360 may be a solid material defining one or more apertures having a sufficient cross-sectional area to allow the passage of air but an insufficient cross-sectional area to allow the passage of the medical fluid F. The distal end 314 of the sidewall 310 has at least one opening to allow air flowing through the cap 380 and the shuttle member 360 to exit the prime tube 300.

During a priming operation, air ejected from the syringe 10A, 10B flows through or around the inner cap 380 and the shuttle member 360, into the internal chamber 350, and out the distal end 314 of the prime tube 300. As the air is continued to be primed and purged out of the syringe 10A, 10B, eventually some medical fluid F pushes the remaining air from the syringe 10A, 10B through the inner cap 380, and the medical fluid F contacts the proximal surface or the proximal post 382 of the inner cap 380. As the medical fluid F is unable to pass through the cap 380, fluid pressure builds until inner cap 380 is dislodged from nozzle 55. The inner cap 380 then slides distally within internal chamber 350 under the force of the medical fluid F as more fluid F is injected into the prime tube 300. The inner cap 380 further engages and pushes the shuttle member 360 within the internal chamber 350 toward the distal end 314.

Similar to embodiments discussed in connection with FIGS. 8A-9C, the shuttle member 360 and/or the inner cap 380 may be a conspicuous color and the sidewall 310 may be at least partially transparent or translucent so that the shuttle member 360 and/or the inner cap 380 are visible within the internal chamber 350. The technologist and/or the controller 900 may determine that the syringe 10A, 10B has been primed based on the position of the shuttle member 360 and/or the inner cap 380 is visible within the internal chamber 350, in substantially the same manner discussed herein in connection with FIGS. 8A-9C.

In some embodiments, the syringe 10A, 10B may be provided with the inner cap 380 already in place. As such, the inner cap 380 may help maintain a sterility of the interior of the syringe 380. Further, as described herein due to the non-manually removable interface between the inner cap 380 from the inner nozzle 55, the inner cap 380 may only be readily removed by a priming operation, thereby ensuring that the syringe 10A, 10B is primed prior to connection of a patient catheter line and minimizing the occurrence of inadvertent injection of air during the injection procedure.

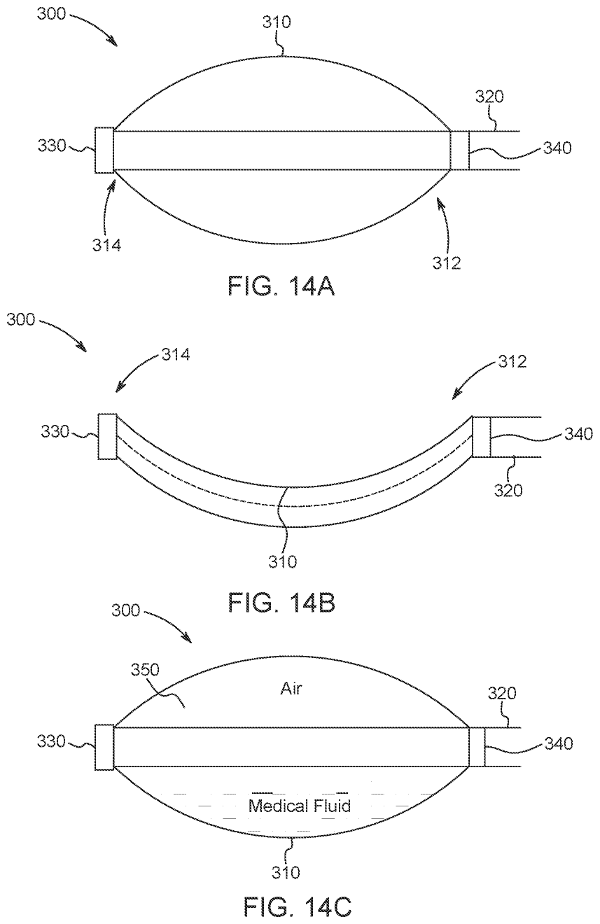

Referring now to FIGS. 14A-14C, an embodiment of a prime tube 300 including an expandable body is illustrated. Similar to the embodiment discussed herein in connection with FIGS. 4A-4D, a closure 330 in the form of a sealing plug may be inserted or otherwise adhered to the distal end 314 of the prime tube 300. The closure 330 may prevent all fluid, including air and medical fluid F, from exiting the distal end 314 of the prime tube 300. In some embodiments, the sidewall 300 may be made of a material which may be blow molded in an expanded state (shown in FIG. 14A) and then converted to a contracted or compressed state (shown in FIG. 14B), which is the state in which the prime tube 300 is supplied. When in the contracted state, the sidewall 310 is at least partially collapsed in on itself such that the volume of the internal chamber 350 is minimized. As shown in FIG. 14A, an embodiment of the prime tube 300 is generally shaped as an ellipsoidal shell as manufactured although other molded shapes are envisioned. After manufacturing, a portion of the sidewall 310 is inverted such that opposite faces of the sidewall 310 touch or come into close proximity with one another. The sidewall 310 as shown, is thus shaped as a half ellipsoidal shell in the contracted state of FIG. 14B. When pressure within the internal chamber 350 exceeds the pressure of the external environment and the compression pressure of the contracted sidewall 310, at least a portion of the sidewall 310 is configured to revert such that the sidewall 310 assumes the expanded state, increasing the volume of the internal chamber 350. In some embodiments, the prime tube 300 may further include a one-way valve 340 that prevents backflow from internal chamber 350 back out the proximal end 312 of the prime tube 300.

The prime tube 300 according to the embodiment of FIGS. 14A-14C operates similarly to the embodiment described in connection with FIGS. 5A-5C. Due to the sealed nature of the sidewall 310 and the closure 330, air is trapped in the internal chamber 350 during the priming operation. As the air is continued to be primed, eventually some medical fluid F pushes the remaining air into the prime tube 300 and the medical fluid F itself enters the internal chamber 350. As the volume of air and medical fluid F in the internal chamber 350 increases, the sidewall 310 inverts to accommodate the increased fluid volume and corresponding pressure. Once the volume of the internal chamber 350 reaches a specific size, the technologist may determine that the syringe 10A, 10B has been primed and is ready for an injection procedure. In some embodiments the priming status sensor 910 may include a camera, as described herein in connection with FIGS. 4A-4D, in conjunction with the controller 900 to determine that the syringe 10A, 10B has been primed based on the sidewall 310 being in the expanded state. As described herein in connection with at least FIGS. 4A-4D, the controller 900 may be programmed or configured to prevent performance of an injection procedure until a successful prime of the syringe 10A, 10B has been detected.

During removal of the prime tube 300 from the syringe 10A, 10B, the one-way valve 340 prevents the pressurized air and fluid in the internal chamber 150 from releasing from the proximal end 312 of the prime tube 300. The entire prime tube 300 with air and medical fluid F contained therein may be discarded according to hospital protocol.

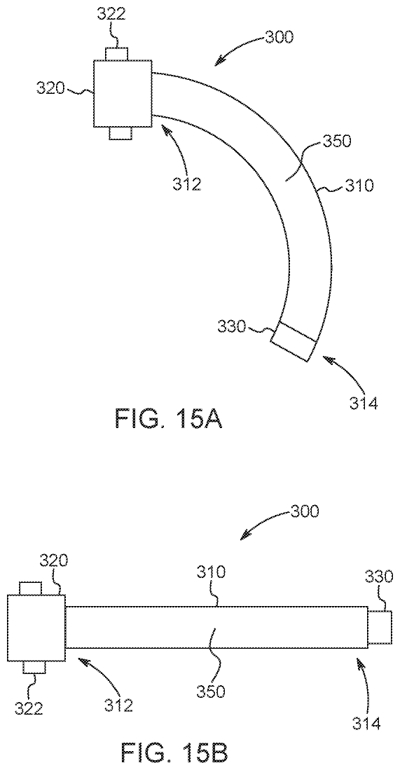

Referring now to FIGS. 15A and 15B, in some embodiments of the prime tube 300, the sidewall 310 may be naturally in a bent, floppy, or flaccid state or configuration, resembling a flattened tube as shown in FIG. 15A. When fluid pressure builds in the internal chamber 350, such when medical fluid F flows through the sidewall 310, the fluid pressure causes the sidewall 310 to straighten/extend and expand to the shape of a tube, as shown in FIG. 15B. In some embodiment, the sidewall 310 may become substantially rigid in response to fluid flow through the internal chamber 350. In other embodiments, the sidewall 310 may be in a flattened, rolled or coiled state in the absence of fluid flow in the internal chamber 350. The sidewall 310 may transition to a straightened expanded shape due to fluid pressure when fluid flows through the internal chamber 350. The closure 330 connected to the distal end 314 of the sidewall 310 may be permeable to air but impermeable to medical fluid F, as described for example in connection with FIGS. 4A-4D herein. The technologist and/or processor 900, as describe herein, may visually determine when the prime tube 300 is in the straightened state (FIG. 15B) and may determine that a priming operation has been completed and may proceed with the next step of the fluid injection procedure. In some embodiments, the closure 300 may define flow features to induce an audible sound where air passes through the closure 330. The technologist may determine that the syringe 10A, 10B has been primed when the aperture 332 ceases to emit a sound, indicating that substantially all of the air has been expelled from the internal chamber 350. Alternatively, the priming status sensor 910 may include a microphone configured to detect the sound emitted from the aperture 332. Controller 900 may be programmed or configured to determine that syringe 10A, 10B has been primed when closure 330 ceases to emit a sound, indicating that substantially all of the air has been expelled from internal chamber 350. Once a successful prime has been established as determined by controller 900 and/or technologist, prime tube 300 may be removed and discarded according to hospital protocol, and a suitably primed patient catheter line may be installed on syringe 10A, 10B.

While various examples of the present disclosure were provided in the foregoing description, those skilled in the art may make modifications and alterations to these examples without departing from the scope and spirit of the disclosure. For example, it is to be understood that features of various embodiments herein may be adapted to other embodiments described herein. Accordingly, the description is intended to be illustrative rather than restrictive. The disclosure is defined by the appended claims, and all changes to the disclosure that fall within the meaning and the range of equivalency of the claims are to be embraced within their scope.

1. A prime tube for use with a fluid injector, the prime tube comprising:
a sidewall defining an internal chamber having an expandable volume;
a connecter associated with a proximal end of the sidewall and configured to reversibly engage an outlet of a fluid reservoir containing a medical fluid; and
a closure associated with a distal end of the sidewall, the closure permeable to air and substantially impermeable to the medical fluid,
wherein the expandable volume of the internal chamber is configured to increase as the medical fluid enters the internal chamber.

2. The prime tube according to claim 1, wherein the sidewall comprises at least one bellows, wherein each of the at least one bellows is configured to transition from a contracted state to an expanded state in response to an increase in fluid pressure within the internal chamber,
wherein the volume defined by the internal chamber is greater with the at least one of the at least one bellows in the expanded state than in the contracted state.

3. (canceled)

4. The prime tube according to claim 2, wherein an axial length of the sidewall is greater with the at least one bellows in the expanded state than in the contracted state.

5. The prime tube according to claim 1, wherein the sidewall comprises an elastomeric material configured to expand the expandable volume in response to an increase in fluid pressure within the internal chamber.

6. The prime tube according to claim 1, wherein the sidewall is configured to transition from a contracted state to an expanded state in response to an increase in fluid pressure within the internal chamber,
wherein, in the contracted state, a distal portion of the sidewall is rolled over a proximal portion of the sidewall,
wherein, in the expanded state, the distal portion of the sidewall is at least partially unrolled from the proximal portion of the sidewall, and
wherein the volume defined by the internal chamber is greater with the sidewall in the expanded state than in the contracted state.

7. (canceled)

8. The prime tube according to claim 6, wherein at least a portion of the sidewall is configured to invert in response to an increase in fluid pressure within the internal chamber to increase the volume of the internal chamber.

9. The prime tube according to claim 1, wherein the sidewall is in a flaccid or bent configuration in the absence of fluid flow through the internal chamber and in a rigid, extended configuration when fluid flows through the internal chamber.

10. The prime tube according to claim 1, wherein the sidewall is in a rolled, coiled configuration in the absence of fluid flow through the internal chamber and in an unrolled, extended configuration when fluid flows through the internal chamber.

11. (canceled)

12. The prime tube according to claim 1, wherein the closure comprises a porous material.

13. The prime tube according to claim 1, wherein the closure defines at least one aperture having a cross-sectional area sized to allow passage of air and substantially prohibit passage of a medical fluid through the at least one aperture.

14. The prime tube according to claim 13, wherein the at least one aperture is configured to emit an audible sound when air flows through the at least one aperture.

15-27. (canceled)

28. A prime tube for use with a fluid injector system, the prime tube comprising:
a sidewall defining an internal chamber having an expandable volume; and
a connecter associated with a proximal end of the sidewall and configured to reversibly engage an outlet of a fluid reservoir containing a medical fluid,
wherein the sidewall is configured to transition from a contracted state to an expanded state in response to an increase in fluid pressure within the internal chamber,
wherein, in the contracted state, a distal portion of the sidewall is rolled over a proximal portion of the sidewall and in the expanded state, the distal portion of the sidewall is at least partially unrolled from the proximal portion of the sidewall, and
wherein the volume defined by the internal chamber is greater with the sidewall in the expanded state than in the contracted state.

29. (canceled)

30. A fluid injector system, comprising:
at least one fluid reservoir configured for injecting a medical fluid;
a prime tube comprising:
a sidewall defining an internal chamber having an expandable volume; and
a connecter associated with a proximal end of the sidewall and configured to reversibly engage an outlet of the at least one fluid reservoir; and
at least one processor programmed or configured to determine a priming status of the prime tube.

31. The fluid injector system according to claim 30, wherein the at least one processor is programmed or configured to determine the priming status of the prime tube based on a measured fluid pressure in at least one of the prime tube and the at least one fluid reservoir.

32. The fluid injector system according to claim 30, further comprising an actuator for injecting the medical fluid from the at least one reservoir, wherein the at least one processor is programmed or configured to determine the priming status of the prime tube based on a measured current draw of the actuator.

33. The fluid injector system according to claim 30, wherein the at least one processor is programmed or configured to determine the priming status of the prime tube based on at least one of expansion of the prime tube and a shape change of the prime tube.

34. (canceled)

35. The fluid injector system according to claim 30, wherein the at least one processor is programmed or configured to determine the priming status of the prime tube based on a force reading on a motor of the fluid injector associated with delivering a fluid from the at least one fluid reservoir.

36. The fluid injector system according to claim 30, wherein the sidewall comprises at least one bellows,
wherein each of the at least one bellows is configured to transition from a contracted state to an expanded state in response to an increase in fluid pressure within the internal chamber, and
wherein the expandable volume defined by the internal chamber is greater with the at least one of the at least one bellows in the expanded state than in the contracted state.

37. The fluid injector system according to claim 30, wherein the sidewall comprises an elastomeric material configured to expand the expandable volume in response to an increase in fluid pressure within the internal chamber.

38. The fluid injector system according to claim 30, wherein the sidewall is configured to transition from a contracted state to an expanded state in response to an increase in fluid pressure within the internal chamber,
wherein, in the contracted state, a distal portion of the sidewall is rolled over a proximal portion of the sidewall and in the expanded state, the distal portion of the sidewall is at least partially unrolled from the proximal portion of the sidewall, and
wherein the expandable volume defined by the internal chamber is greater with the sidewall in the expanded state than in the contracted state.

39-44. (canceled)
